(19) United States
(12) Patent Application Publication
(10) Pub. No.: US 2003/0091591 A1
(43) Pub. Date: May 15, 2003

(54) Alphavirus expression vectors and uses thereof

(75) Inventors: Cheng Xiong, Fort Collins, CO (US); Robert B. Grieve, Windsor, CO (US)

Correspondence Address: HESKA CORPORATION, INTELLECTUAL PROPERTY DEPT., 1613 PROSPECT PARKWAY, FORT COLLINS, CO, 80525, US

(73) Assignee: HESKA CORPORATION

(21) Appl. No.: 10/251,470

(22) Filed: Sep. 20, 2002

Related U.S. Application Data
(63) Continuation of application No. 08/501,017, filed on Aug. 8, 1995.
Application No. 08/501,017 is a 371 of international application No. PCT/US94/01398, filed on Feb. 8, 1994.
Application No. PCT/US94/01398 is a continuation-in-part of application No. 08/015,414, filed on Feb. 8, 1993, now abandoned.

(51) Int. Cl. A61K 39/12; C12N 7/00; C12N 15/86

(52) U.S. Cl. 424/199.1; 435/235.1; 435/456

(57) ABSTRACT

The present invention relates to a recombinant virus particle vaccine comprising a recombinant molecule packaged in an alphavirus coat. A preferred recombinant molecule to incorporate into such a virus particle is one that encodes a protective compound (e.g. a protective protein or a protective RNA) capable of protecting an animal from a disease, such that the nucleic acid sequence is operatively linked to a packaging-defective alphavirus expression vector that is capable of directing replication and transcription of the recombinant molecule. The invention also includes methods to produce and use such vaccines to protect animals from disease, particularly from disease caused by protozoan parasites such as T. gondii, hehnlinth parasites, ectoparasites, fungi, bacteria, or viruses. The present invention also includes recombinant molecules having an alphavirus expression vector capable of directing the expression of at least one compound capable of protecting an animal from parasitic disease when the recombinant molecule is transfected into an animal cell. Also included is the use of such recombinant molecules to rapidly and efficiently produce, in eukaryotic cells, compounds protective against parasitic infection.

### FIELD OF THE INVENTION

The present invention relates to novel recombinant live alphavirus vaccines and their use to protect animals from, for example, infectious or metabolic diseases. The present invention also relates to the use of alphavirus-based expression systems to produce such vaccines and other compounds capable of protecting animals from disease, including diseases caused by parasites, such as toxoplasmosis and heartworm.

### BACKGROUND OF THE INVENTION

Despite intense efforts by laboratories throughout the world, there are currently essentially no effective vaccines against a number of infectious and metabolic diseases, including most, if not all, parasitic diseases. Although some promising antigens have been identified, there remains a need for an improved method to deliver efficacious protective compounds to an animal. In addition, there remains a need for a rapid, efficient and cost-effective method to produce efficacious protective compounds.

Live vaccines have been associated with longer-lasting immunity than inactivated vaccines. However, one disadvantage of live vaccines has been their ability to revert to virulence. In an attempt to overcome this problem, several viral and bacterial systems, such as poxviruses, herpesviruses, adenoviruses, Salmonella, and BCG (Bacillus Calmette-Guerin), have been genetically manipulated to generate vectors containing heterologous antigen genes in order to immunize a host with a vaccine in which the antigens are presented in a “live” configuration (i.e., in which the antigens are exposed on the outside of a cell membrane or viral coat). See, for example, the following two review articles: Esposito et al., pp. 195-247, 1989, Advances in Veterinary Science and Comparative Medicine, Vol. 33; Dougan et al., pp. 271-300, 1989, Advances in Veterinary Science and Comparative Medicine, Vol. 33. However, none of these systems has yet been commercialized.

Alphaviruses, which are members of the togavirus family, are attractive vaccine carriers because they have a wide host range. Alphavirus-based expression vectors are also attractive for the production of protective compounds in eukaryotic cells because alphaviruses have a wide host range, function in the cytoplasm and are capable of directing rapid and effective expression of viral structural genes under the control of alphavirus subgenomic promoters. Although some alphaviruses, such as Semliki Forest virus, are pathogenic, Sindbis virus has not been associated with natural disease in humans or animals. The genomes of several alphaviruses, including Sindbis virus, Semliki Forest virus, and Ross River virus, Venezuela equine encephalitis virus have been cloned and their nucleotide sequences determined; see, for example, Strauss et al., pp. 91-110, 1984, Virology, Vol. 133; Liljestrom et al., pp. 4107-4113, 1991, J. Virology, Vol. 65; and Faragher et al., pp. 509-526, Virology, Vol. 163. FIG. 1 presents a schematic drawing of the location of the genes encoding the nonstructural and structural polypeptides of alphaviruses as well as of the transcription control regions, including the subgenomic promoter, that regulate the gene expression.

Both Sindbis and Semliki Forest viral expression vectors have been used to produce certain heterologous proteins in cell culture. The use of small Sindbis virus defective interfering (DI) RNA-based vectors, however, has been relatively ineffective; see, for example, Bredenbeek et al., pp. 297-310, 1992, Seminars in Virology, Vol. 3. Larger Sindbis virus vectors have been used to produce chloramphenicol acetyltransferase and tissue plasminogen activator (Xiong et al., pp. 1188-1191, 1989, Science, Vol. 243; Huang et al., pp. 85-91, 1989, Virus Genes, Vol. 3), and Semliki Forest virus vectors have been used to produce the human transferrin receptor, mouse dihydrofolate reductase, chick lysozyme, and beta-galactosidase (Liljestrom et al., pp. 1356-1361, 1991, Bio/Technology, Vol. 9). Several other proteins have been expressed transiently; see review by Bredenbeek et al., ibid. Liljestrom et al., ibid., however, state that technical difficulties (e.g., low transfection rates) have precluded wide spread use of Sindbis virus vectors. Furthermore, the inventors are not aware of the use of alphavirus-based expression vectors to produce any compounds capable of protecting animals from parasitic infections, such as those caused by protozoa, helminths, ectoparasites and/or fungi. Neither are the inventors are unaware of the use of a live alphavirus-based recombinant vaccine to protect an animal from infectious or metabolic diseases.

As such, there is a need for new and improved recombinant methods to produce efficacious protective compounds as well as a need for an improved vaccine delivery system to protect animals from metabolic disorders or infectious agents.

### SUMMARY OF THE INVENTION

The present invention includes a new method to protect animals from disease using a recombinant virus particle vaccine comprising a packaging-defective recombinant molecule packaged in an alphavirus coat. When the recombinant virus particle vaccine of the present invention is administered to an animal, the virus particle is able to infect cells within the animal. Infected cells are able to express nucleic acid sequences present on the packaging-defective recombinant molecule to produce compounds capable of protecting the animal from a variety of diseases. Using methods taught in the present invention, vaccines can be generated that are capable of protecting an animal from any disease for which a protective protein or protective RNA species can be produced. As such, the present invention is of extremely broad scope and includes a wide variety of vaccines that have a variety of applications.

One embodiment of the present invention is a recombinant virus particle vaccine that includes a packaging-defective recombinant molecule packaged in an alphavirus coat, the vaccine being capable of protecting an animal from disease when administered to the animal in an effective amount. The packaging-defective recombinant molecule includes a nucleic acid sequence that encodes a protective compound, such as a protective protein or protective RNA species, operatively linked to a packaging-defective alphavirus expression vector that is capable of directing expression, and preferably also replication, of the packaging-defective recombinant molecule. Animals administered the vaccine are able to produce the protective compound encoded by the packaging-defective recombinant molecule of the vaccine and thereby are protected from any disease that the protective compound can effectively neutralize or otherwise counteract.

Upon administration to an animal, preferred vaccines of the present invention are capable of effecting production of a protective protein that can elicit an immune response to protect the animal from, for example, an infectious agent. Other preferred vaccines are capable of eliciting production of antisense RNA molecules to protect an animal from disease. Particularly preferred vaccines contain nucleic acid sequences that encode at least one antigen, preferably Toxoplasma gondii P30 or a functional equivalent thereof, capable of eliciting an immune response to protect an animal from toxoplasmosis.

The present invention also relates to a method for protecting an animal from disease by administering to such an animal an effective amount of a recombinant virus particle vaccine of the present invention. Vaccines can be administered in a variety of ways and can, but need not, include an immunopotentiator. According to one embodiment, a protective protein containing amino acids encoded by the nucleic acid sequence contained in the virus particle is also administered to the animal either prior to, following, or both prior to and following administration of the recombinant virus particle vaccine in order to enhance the immunogenic response.

The present invention also relates to a method to produce recombinant virus particle vaccines, which includes transfecting a packaging-defective recombinant molecule of the present invention into a host cell, preferably a mammalian, insect, or avian cell, in such a manner that culturing of the transfected cell yields recombinant virus particles. For example, a packaging-defective recombinant molecule can be co-transfected into a host cell with an alphavirus packaging vector that is capable of effecting packaging of the packaging-defective recombinant molecule into a virus particle, but is essentially incapable of self-packaging. Alternatively, the host cell to be transfected by the packaging-defective recombinant molecule can already contain the genetic information required to effect packaging of the packaging-defective recombinant molecule into a virus particle. Transfected cells are subsequently cultured to produce virus particles, which are then recovered and formulated into a vaccine.

The present invention also includes a rapid and efficient method to produce, in a eukaryotic cell, compounds that are effective in protecting animals from parasitic infection. As such, one embodiment of the present invention is a therapeutic composition capable of protecting an animal from disease caused by a parasite when the composition is administered to the animal in an effective amount. In accordance with the embodiment, the composition is produced by a method that includes the steps of (a) culturing an animal cell transfected with a recombinant molecule to produce a compound capable of protecting the animal from the disease, (b) recovering the protective compound, and (c) formulating a therapeutic composition therefrom. Such a recombinant molecule includes at least one nucleic acid sequence encoding a protective compound operatively linked to an alphavirus expression vector. The recombinant molecule can be either packaging-defective or packaging-competent. Also included in the present invention is a method to protect an animal from a disease caused by a parasite that includes administering to the animal an effective amount of a therapeutic composition produced as disclosed above.

Another aspect of the present invention involves recombinant molecules and the production thereof. Recombinant molecules of the present invention can be generated by a method that includes the steps of (a) producing a nucleic acid sequence encoding a protective compound, (b) producing an alphavirus expression vector capable of directing expression (and preferably also replication) of the recombinant molecule, and (c) operatively linking the nucleic acid sequence of (a) to the expression vector of (b) to obtain a recombinant molecule in which expression of the nucleic acid sequence is controlled by the expression vector. A recombinant molecule of the present invention is capable of directing expression of at least one protective compound when the recombinant molecule is transfected into an animal host cell. Such a recombinant molecule includes at least one nucleic acid sequence encoding the protective compound operatively linked to an alphavirus expression vector, the compound being capable of protecting an animal from disease, preferably from a disease caused by a parasite. Either a packaging-defective or packaging-competent recombinant molecule can be used to produce protective compounds by culturing recombinant cells containing such recombinant molecules. Packaging-defective recombinant molecules are preferably used in the production of recombinant virus or recombinant cell therapeutic compounds of the present invention. Packaging-defective recombinant molecules are used in recombinant virus particle vaccines of the present invention.

The invention also includes a recombinant molecule that includes an alphavirus expression vector operatively linked to a nucleic acid sequence encoding a fusion protein, the fusion protein comprising a fusion segment joined to a protein heterologous to the alphavirus. Preferably the fusion protein also includes a signal segment capable of promoting secretion of the fusion protein.

Another embodiment is a recombinant molecule that includes a nucleic acid sequence encoding at least one antigen operatively linked to an alphavirus expression vector. The antigen, preferably a Toxoplasma antigen, a Dirofilaria antigen or a combination thereof, also includes a signal segment capable of secreting the antigen from an animal cell transfected by the recombinant molecule.

The present invention also includes a recombinant cell capable of producing at least one compound capable of protecting an animal from disease caused by a parasite. Such a recombinant cell comprises an animal cell transfected with a recombinant molecule capable of directing expression of the protective compound. Also included is a method to produce the protective compound that includes the steps of (a) culturing a recombinant cell of the present invention (i.e., an animal cell transfected with a recombinant molecule (i.e., a recombinant cell) to produce the protective compound, and (b) recovering the protective compound.

Expression of nucleic acid sequences of the present invention is effected by alphavirus expression vectors to which the nucleic acid sequences are operatively linked. Preferred alphavirus expression vectors include Sindbis virus expression vectors, Semliki Forest virus expression vectors, Ross River virus expression vectors, Venezuela equine encephalitis virus and hybrids thereof, with Sindbis virus expression vectors being more preferred. According to one embodiment, a nucleic acid sequence of the present invention is operatively linked to an alphavirus subgenomic promoter. Alphavirus expression vectors include packaging defective and packaging-competent alphavirus expression vectors.

Nucleic acid sequences of the present invention can encode one or more protective compounds of the present invention. Nucleic acid sequences of the present invention can be engineered to permit protective compounds produced by infected or otherwise transfected cells to remain inside the cell, to be secreted from the cell, or to be attached to the outer cell membrane. Preferred nucleic acid sequences are those that encode compounds capable of protecting an animal from a variety of diseases, such as those listed below. Particularly preferred nucleic acid sequences are those that encode compounds capable of protecting an animal from toxoplasmosis or heartworm.

One embodiment of the present invention is a nucleic acid sequence that encodes one of the following modified T. gondii P30 antigens: (a) a P30 antigen lacking amino terminal hydrophobic residues, (b) a P30 antigen lacking carboxyl terminal hydrophobic residues, or (c) a P30 antigen lacking both amino terminal and carboxyl terminal hydrophobic residues. Also included are the proteins encoded by such nucleic acid sequences.

Preferred recombinant virus particle vaccines or other therapeutic compositions of the present invention are those that protect animals from diseases caused by protozoan parasites, helminth parasites, ectoparasites, fungi, bacteria, and viruses. Particularly preferred are those vaccines that protect animals from disease caused by infectious agents of the genera Toxoplasma, Dirofilaria, Acanthocheilonema, Babesia, Brugia, Candida, Cryptococcus, Cryptosporidium, Dipetalonema, Eimeria, Encephalitozoon, Hepatozoon, Histoplasma, Isospora, Loa, Microsporidia, Neospora, Nosema, Onchocerca, Parafilaria, Plasmodium, Pneumocystis, Rochalimaea, Setaria, Stephanofilaria, Theileria and Wuchereria; and even more particularly, vaccines that protect animals against infection by T. gondii, Dirofilaria immitis, and/or Cryptosporidium.

A preferred embodiment of the present invention is a method to protect an animal from toxoplasmosis by administering to the animal an effective amount of a recombinant virus particle vaccine. The recombinant virus particle vaccine includes a recombinant molecule containing a nucleic acid sequence encoding a T. gondii P30 antigen or functional equivalent of the antigen, the nucleic acid sequence being operatively linked to a packaging-defective Sindbis virus expression vector capable of directing replication and expression of the recombinant molecule. The vaccine, when administered to an animal in an effective amount, is preferably capable of infecting the animal so as to cause the production of P30 antigen which subsequently acts to elicit an immune response capable of protecting the vaccinated animal from toxoplasmosis.

Another preferred method to protect an animal from toxoplasmosis includes administering to the animal an effective amount of a therapeutic composition produced by a method including the steps of (a) culturing an animal cell transfected with a recombinant molecule to produce a T. gondii P30 antigen, (b) recovering the antigen, and (c) formulating a therapeutic composition therefrom.

### BRIEF DESCRIPTION OF THE FIGURES

FIG. 1 is a schematic drawing depicting an alphavirus RNA genome and subgenomic RNA.

FIG. 2 depicts a hydrophilicity plot of T. gondii P30 antigen.

FIG. 3 includes schematic drawings of several nucleic acid sequences that encode modified T. gondii P30 antigens.

FIG. 4 includes schematic drawings of Sindbis virus expression vectors SV1 and SV2 as well as a schematic drawing of a full-length Sindbis virus vector.

FIG. 5 schematically depicts the derivation of Sindbis virus packaging vector PV1.

### DETAILED DESCRIPTION OF THE INVENTION

The present invention includes a recombinant virus particle vaccine, methods to produce the vaccine, and methods to use the vaccine, preferably to immunize animals against infectious or metabolic diseases.

A recombinant virus particle vaccine of the present invention includes recombinant genetic information packaged in a viral coat. As such, a recombinant virus particle vaccine of the present invention is essentially a live vaccine and is advantageous because live vaccines are believed to confer more vigorous and longer-lasting immunity. While not being bound by theory, it is believed that such advantages are due to the ability of the genetic information carried by the virus particle vaccine to enter the cells of the vaccinated animal, replicate itself, and direct the expression of a protective compound, such as a protective protein (e.g., an immunogenic protein, including carbohydrate, amino acid and/or other epitopes) or a protective RNA species (e.g., an antisense RNA species) for extended periods of time. Thus, such a vaccine need not be administered frequently and, in fact, the virus particle vaccine essentially can function as a self-booster.

In addition, since the genetic information is packaged in a viral coat, the genetic information is protected from degradation and is injected into host cells by the normal route of infectivity, that is through normal cellular receptors that recognize and take up viruses. Recombinant virus particle vaccines of the present invention have a wide host range by virtue of their being able to infect multiple species and cell types. Moreover, since the genetic information present in the vaccine of the present invention is packaging-defective (i.e., unable to effect packaging of the genetic information into a viral coat), essentially no infectious virus is produced by the infected cells.

A recombinant viral particle vaccine of the present invention is capable of delivering to a vaccinated animal a nucleic acid sequence of the present invention plus the proper alphavirus regulatory sequences so that the vaccinated animal can produce a protective compound encoded by that nucleic acid sequence. Essentially any nucleic acid sequence that encodes a protective compound, such as a protective protein or protective RNA species can be used in the present invention. As such, it is within the scope of the present invention to develop recombinant virus particle vaccines against a variety of infectious diseases such as those caused by protozoan parasite, helminth parasite, ectoparasite, fungal, bacterial, or viral infectious agents. It is also within the scope of the present invention to develop vaccines against a variety of metabolic diseases, such as Cushing's disease or cancer preferably using a vaccine encoding a protective RNA species.

A recombinant virus particle vaccine of the present invention includes a packaging-defective recombinant molecule packaged in an alphavirus coat. As used herein, an “alphavirus coat” is a viral coat containing at least one alphavirus structural polypeptide.

A recombinant molecule of the present invention includes an alphavirus expression vector which is operatively linked to a nucleic acid sequence encoding a protective compound of the present invention. A recombinant molecule of the present invention can be either packaging-competent or packaging-defective. A packaging-competent recombinant molecule includes an alphavirus expression vector that contains the genes and regulatory sequences necessary to effect packaging of the recombinant molecule into a virus (i.e., a packaging-competent alphavirus expression vector). A packaging-defective recombinant molecule includes an alphavirus expression vector that is essentially incapable of effecting packaging of the packaging-defective recombinant molecule into a virus (i.e., a packaging-defective alphavirus expression vector). The present invention includes RNA recombinant molecules as well as DNA recombinant molecules from which the RNA recombinant molecules can be transcribed. As used herein, “operatively linked” means that the nucleic acid sequence is joined (i.e., ligated) to the alphavirus expression vector in such a manner that regulatory signals present on the vector (e.g., promoters) lead to the expression of the nucleic acid sequence, ultimately leading to the production of the corresponding protective compound (i.e., the compound encoded by the nucleic acid sequence).

As used herein, a protective compound is a compound that is able to treat, ameliorate, or prevent disease, such as that caused by an infectious agent (e.g., a parasite) or by a metabolic disorder. Protective compounds include protective proteins and protective RNA species. As used herein, a “protective protein” is a protein that, when produced by an animal administered a recombinant virus particle vaccine of the present invention or when produced in cell culture and subsequently administered to an animal, is able to treat, ameliorate, or prevent an infectious or metabolic disease in that animal. Similarly, a “protective RNA species”, or a “protective RNA”, is an RNA molecule that, when produced by an animal administered a recombinant virus particle vaccine of the present invention or when produced in cell culture and subsequently administered to an animal, is able to treat, ameliorate, or prevent an infectious or metabolic disease in that animal.

As used herein, the phrases “to protect an animal from disease” and “to protect an animal from infection” refer to the ability of a protective compound of the present invention to treat, ameliorate, or prevent an infectious disease in an animal administered in an effective amount the compound and/or a virus particle vaccine capable of producing the compound, by for example, interfering with the infectious agent that causes the disease. Similarly, the phrase “to protect an animal from metabolic disease” refers to the ability of the protective protein or RNA species to treat, ameliorate, or prevent a metabolic disease in an animal administered an effective amount of the protective compound and/or a virus particle vaccine capable of producing the compound.

A protective protein of the present invention can be, for example, an immunogen that elicits an immune response capable of protecting an animal from the corresponding infectious disease. A protective protein can also be some other compound, such as a toxin, enzyme, antibody, or other binding protein, that is capable of neutralizing the infectious agent which causes the disease. A preferred protective protein of the present invention is an immunogen capable of eliciting both humoral and cell-mediated immunity to protect the animal from the disease. As used herein, protective proteins can include full-length proteins as well as modified versions of the protein in which amino acids have been deleted (e.g., a truncated version of the protein, such as a peptide), inserted, inverted, substituted and/or derivatized, such as by post-translational modification (e.g., glycosylation, phosphorylation, acetylation, carboxyl-terminal amidation) such that the modified version of the protein has a biological function substantially similar to that of the natural protein in its ability to protect an animal from disease (i.e., functionally equivalent to the natural protein). Modifications can be accomplished by techniques known in the art including, but not limited to, direct modifications to the protein or modifications to the nucleic acid sequence encoding the protein using, for example, classic or recombinant DNA techniques to effect random or targeted mutagenesis. See, for example, Sambrook et al., Molecular Cloning: A Laboratory Manual, Cold Spring Harbor Labs Press, 1989. Protective proteins of the present invention, including modified versions thereof, can be identified in a straight-forward manner using any one of a number of screening techniques known to those skilled in the art including, but not limited to, functional assays and binding assays. In one embodiment, a modified, functionally equivalent T. gondii antigen can be selected by its ability to elicit an immune response capable of protecting an animal from toxoplasmosis.

Protective protein immunogens of the present invention are of a sufficient size to form an epitope, a size that is typically at least about 7 to about 9 amino acids in length. As is appreciated by those skilled in the art, an epitope can include amino acids that naturally are contiguous to each other as well as amino acids that, due to the tertiary structure of the natural protein, are in sufficiently close proximity to form an epitope. Such epitopes can be identified, for example, by mutation analysis or by analysis of the three-dimensional structure of epitopes comprising an immunogen.

Protective proteins of the present invention can also include mimetopes, which include any compound that is able to mimic the ability of a protective protein of the present invention to protect an animal from parasitic disease. A mimetope can be a peptide that has been modified to decrease its susceptibility to degradation (for example, by replacing a scissile peptide bond with a bond that cannot be efficiently cleaved by the endoprotease) but that still retains its protective ability. Other examples of mimetopes include, but are not limited to, anti-idiotypic antibodies, or fragments thereof, that include at least one binding site that mimics one or more epitopes of a protective protein; and nucleic acids, that have a structure similar to at least one epitope of a protective protein of the present invention.

A protective RNA species of the present invention can be, for example, a recombinant molecule encoding a protective protein or RNA, an antisense RNA species, a ribozyme or an RNA-based drug that, when administered to an animal in an effective amount, is capable of protecting the animal from disease, and preferably from parasitic disease. Antisense RNA species and/or ribozymes can be used to reduce or prevent expression of parasitic genes, thereby protecting an animal from disease. As used herein, an RNA-based drug is any RNA molecule that is of sufficient size and/or structure to be able to interact with an intra- or extra-cellular component in order to prevent, treat, or ameliorate a disease otherwise caused by that component. Protective RNA species include RNA species that have been modified to, for example, increase their stability without substantially affecting the protective function of the RNA. For example, the backbone of an RNA species can be modified to decrease the susceptibility of the backbone to nucleases.

An isolated nucleic acid sequence of the present invention refers to any DNA or RNA molecule having a nucleic acid sequence that encodes a protective protein or RNA species and that has been separated from its natural milieu. A nucleic acid sequence of the present invention can be isolated from natural sources, can be obtained by mutating natural isolates using classic or recombinant DNA techniques, or can be synthesized chemically.

Nucleic acid sequences of the present invention include those that encode naturally-occurring (i.e., native) protective proteins as well as those that encode functional equivalents thereof (i.e., functionally equivalent protective compounds). As used herein, a “functionally equivalent” protective protein is a protein that has substantially the same biological activity as the naturally-occurring protective protein; that is, the functionally equivalent protective protein is capable of protecting an animal from infectious disease. Nucleic acid sequences that encode functionally equivalent protective proteins are herein referred to as functionally equivalent nucleic acid sequences and-include nucleic acid sequences having deletions, additions, inversions, and/or substitutions which, in spite of the modifications, encode protective proteins. The minimal size of a functionally equivalent nucleic acid sequence is the shortest length of nucleotides required to encode a protein capable of protecting an animal from an infectious disease.

Additional nucleic acid sequences of the present invention include nucleic acid sequences that encode RNA-based drugs, antisense RNA species or ribozymes. As used herein, an “RNA-based drug” is any RNA molecule that is of sufficient size and/or structure to be able to interact with an intra- or extra-cellular component in order to prevent, treat, or ameliorate a disease otherwise caused by that component. As used herein an “antisense RNA” or “antisense RNA species” is any RNA molecule that is capable of substantially preventing expression of a detrimental protein. As such, a nucleic acid sequence encoding such an RNA can be of any size and structure that, when expressed, will yield an antisense RNA having the defined function.

A functionally equivalent nucleic acid sequence can be obtained using methods known to those skilled in the art. See, for example, Sambrook et al., Molecular Cloning: A Laboratory Manual, Cold Spring Harbor Labs Press, 1989. For example, nucleic acid sequences can be modified using a variety of techniques including, but not limited to, classic mutagenesis techniques and recombinant DNA techniques, such as site-directed mutagenesis, chemical treatment of a nucleic acid to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid sequences, and combinations thereof. Functionally equivalent nucleic acids can be selected from a mixture of modified nucleic acid sequences by screening for the function of the protein or antisense RNA encoded by the nucleic acid sequence. A number of screening techniques are known to those skilled in the art including, but not limited to, functional assays and binding assays. In one embodiment, a nucleic acid sequence that encodes a functionally equivalent T. gondii antigen can be selected by its ability to encode a protein capable of eliciting an immune response that protects an animal from toxoplasmosis.

The present invention particularly involves recombinant virus particle vaccines that protect animals from infectious agents such as protozoan parasites, helminth parasites, ectoparasites, fungi, bacteria and viruses. As used herein, Microsporidia organisms are classified as protozoan parasites. As used herein, Pneumocystis organisms are classified as fungi, although there is still controversy as to whether or not they should be classified as protozoa or fungi. Preferably, recombinant virus particle vaccines of the present invention protect animals from protozoan parasites, helminth parasites (such as nematodes, cestodes and trematodes, with filarial, ascarid, strongyle and trichostrongyle nematodes being more preferred), ectoparasites, and/or fungi such as those that cause heartworm, malaria, coccidiosis, toxoplasmosis, or other AIDS-related opportunistic infections. More preferably, the vaccine is effective against at least one parasite of the genus Toxoplasma, Dirofilaria, Acanthocheilonema, Babesia, Brugia, Candida, Cryptococcus, Cryptosporidium, Dipetalonema, Eimeria, Encephalitozoon, Hepatozoon, Histoplasma, Isospora, Loa, Microsporidia, Neospora, Nosema, Onchocerca, Parafilaria, Plasmodium, Pneumocystis, Rochalimaea, Setaria, Stephanotilaria, Theileria and Wuchereria. The vaccine is even more preferably effective against T. gondii, D. immitis, or Cryptosporidium parasites. Other suitable parasites against which to develop a vaccine include Aelurostrongylus abstrusus, Ancylostoma spp., Angiostrongylus spp., Brugia malayi, Bunostomum spp., Chabertia ovina, Cooperia spp., Dictyocaulus spp., Dipetalonema streptocerca, Dipetalonema perstans, Encephalitozoon cuniculi, Encephalitozoon hellem, Enterobius vermicularis, Filaroides spp., Haemonchus spp., Loa loa, Mansonella ozzardi, Nematodirus spp., Nosema corneum, Oesophagostomum spp., Onchocerca volvulus, ostertagia spp., Pneumocystis carinii, Strongyloides spp., Strongylus spp., Trichinella spiralis, Trichostrongylus spp., Trichuris spp., Uncinaria spp., Wuchereria bancrofti, those nematodes of the order Ascaridida (Ascarids) and Cyathostominae (small strongyles of horses).

Preferred nucleic acid sequences of the present invention are those that encode protective compounds that protect animals from infectious diseases caused by protozoan parasites, helminth parasites, ectoparasites, fungi, bacteria or viruses. More preferred nucleic acid sequences are those that encode protective proteins that protect animals from protozoan parasites, helminth parasites, ectoparasites, and/or fungi such as those that cause heartworm, malaria, coccidiosis, toxoplasmosis, or other AIDS-related opportunistic infections.

A particularly preferred nucleic acid sequence of the present invention encodes a T. gondii antigen that is capable of eliciting an immune response to protect an animal from infection by T. gondii, which is a parasite that causes toxoplasmosis. T. gondii antigens useful in the present invention include, but are not limited to, tachyzoite antigens P30, P23, and P22, and other T. gondii antigens with molecular weights of about 25, about 28, about 30, about 35, about 41, about 54, about 66, and about 68 kilodaltons. As used herein, a T. gondii P30 antigen refers to a protein corresponding to the natural antigen as well as to proteins comprising modified forms of a natural P30 antigen capable of protecting an animal from toxoplasmosis. Similarly, any other antigen of the present invention referred to as “an” antigen includes a protein corresponding to the natural antigen as well as to modifications thereof capable of protecting an animal from the corresponding disease. Such modifications, or functional equivalents, can be isolated using methods taught herein; see, for example the Examples.

Additional particularly preferred nucleic acid sequences of the present invention encode at least one of the following proteins: D. immitis P39, D. immitis P22L, D. immitis P22U, D. immitis P20.5, D. immitis P4, D. immitis Di22 and/or D. immitis proteases expressed in L3 and/or L4 larvae, as well as other helminth proteins sharing significant homology with such D. immitis proteins. A protein sharing significant homology with another protein refers to the ability of the nucleic acid sequences encoding such proteins to form stable hybridization complexes with each other under stringent hybridization conditions, as described, for example, in Sambrook et al., ibid. Grieve et al., in PCT International Publication No. WO 92/13560, published Aug. 20, 1992, disclose a method to identify D. immitis antigens capable of selectively binding to at least one component of immune serum that is capable of inhibiting heartworm development. U.S. patent application Ser. No. 08/003,389, filed Jan. 12, 1993, entitled “Immunogenic Larval Proteins”, discloses a 39-kD D. immitis protein (size determined by Tris glycine SDS-PAGE (sodium dodecyl sulfate polyacrylamide gel electrophoresis)), referred to herein as P39, and a nucleic acid sequence that encodes it. U.S. patent application Ser. No. 08/003,257, filed Jan. 12, 1993, entitled “Reagents and Methods for Identification of Vaccines”, discloses 22-kD and 20.5-kD D. immitis proteins (sizes determined by Tris glycine SDS-PAGE), referred to herein as P22L and P20.5, and nucleic acid sequences that encode them. U.S. patent application Ser. No. 08/109,391, filed Aug. 19, 1993, entitled “Novel Parasitic Helminth Proteins”, discloses D. immitis P4 and D. immitis P22U, as well as nucleic acid sequences that encode them. U.S. patent application Ser. No. 08/060,500, filed May 10, 1993, entitled “Heartworm Vaccine”, discloses a D. immitis Di22 protein and a nucleic acid sequence encoding it (included in GeneBank data base accession number M82811); Ser. No. 08/060,500 is a continuation of U.S. patent application Ser. No. 07/683,202, filed Apr. 8, 1991. U.S. patent application Ser. No. 08/153,554, filed Nov. 16, 1993, entitled “Protease Vaccine Against Heartworm”, discloses D. immitis larval proteases; Ser. No. 08/153,554 is a continuation of U.S. patent application Ser. No. 07/792,209, filed Nov. 12, 1991.

A particularly preferred nucleic acid sequence for use in the present invention is one encoding a protective protein corresponding to the T. gondii P30 antigen, the major surface antigen of the tachyzoite stage of T. gondii infection. P30 is advantageous because it has been shown to protect mice from virulent T. gondii challenge (Khan et al., pp. 3501-3506, 1991, J. Immunol., Vol. 147; Bulow et al., pp. 3496-3500, 1991, J. Immunol., Vol. 147). The supply of native P30, however, has to date been too limited to make the isolation and use of such a native protein as a vaccine feasible. As such, the gene encoding the P30 antigen, which has been isolated and sequenced by Burg et al., pp. 3584-3591, 1988, J. Immunol., Vol. 141. is particularly useful in the present invention. Table 1 corresponds to the coding region, and deduced amino acid sequence, of a full-length primary translation product of the T. gondii P30 antigen.

TABLE 1
Primary Translation Product of the T. gondii P30 Antigen

| | |
|---|---|
| ATG TCG GTT TCG CTG CAC CAC TTC ATT ATT TCT TCT GGT TTT TTG ACG | 48 |
| Met Ser Val Ser Leu His His Phe Ile Ile Ser Ser Gly Phe Leu Thr | |
| 1 5 10 15 | |
| ACT ATG TTT CCG AAC GCA GTG AGA CGC GCC GTC ACG GCA GGG GTG TTT | 96 |
| Ser Met Phe Pro Lys Ala Val Arg Arg Ala Val Thr Ala Gly Val Phe | |
| 20 25 30 | |
| GCC GCG CCC ACA CTG ATG TCG TTC TTG CGA TGT GGC GTT ATG GCA TCG | 144 |
| Ala Ala Pro Thr Leu Met Ser Phe Leu Arg Cys Gly Val Met Ala Ser | |
| 35 40 45 | |
| GAT CCC CCT CTT GTT GCC AAT CAA GTT GTC ACC TGC CCA GAT AAA AAA | 192 |
| Asp Pro Pro Leu Val Ala Asn Gln Val Val Thr Cys Pro Asp Lys Lys | |
| 50 55 60 | |
| TCG ACA GCC GCG GTC ATT CTC ACA CCG ACG GAG AAC CAC TTC ACT CTC | 240 |
| Ser Thr Ala Ala Val Ile Leu Thr Pro Thr Glu Asn His Phe Thr Leu | |
| 65 70 75 80 | |
| AAG TGC CCT AAA ACA GCG CTC ACA GAG CCT CCC ACT CTT GCG TAC TCA | 288 |
| Lys Cys Pro Lys Thr Ala Leu Thr Glu Pro Pro Thr Leu Ala Tyr Ser | |
| 85 90 95 | |
| CCC AAC AGG CAA ATC TGC CCA GCG GGT ACT ACA AGT AGC TGT ACA TCA | 336 |
| Pro Asn Arg Gln Ile Cys Pro Ala Gly Thr Thr Ser Ser Cys Thr Ser | |
| 100 105 110 | |
| AAG GCT GTA ACA TTG AGC TCC TTG ATT CCT GAA GCA GAA GAT AGC TGG | 384 |
| Lys Ala Val Thr Leu Ser Ser Leu Ile Pro Glu Ala Glu Asp Ser Trp | |
| 115 120 125 | |
| TGG ACG GGG GAT TCT GCT AGT CTC GAC ACG GCA GGC ATC AAA CTC ACA | 432 |
| Trp Thr Gly Asp Ser Ala Ser Leu Asp Thr Ala Gly Ile Lys Leu Thr | |
| 130 135 140 | |
| GTT CCA ATC GAG AAG TTC CCC GTG ACA ACG CAG ACG TTT GTG GTC GGT | 480 |
| Val Pro Ile Glu Lys Phe Pro Val Thr Thr Gln Thr Phe Val Val Gly | |
| 145 150 155 160 | |
| TGC ATC AAG GGA GAC GAC GCA CAG AGT TGT ATG GTC ACG GTG ACA GTA | 528 |
| Cys Ile Lys Gly Asp Asp Ala Gln Ser Cys Met Val Thr Val Thr Val | |
| 165 170 175 | |
| CAA GCC AGA GCC TCA TCG GTC GTC AAT AAT GTC GCA AGG TGC TCC TAC | 576 |
| Gln Ala Arg Ala Ser Set Val Val Asn Asn Val Ala Arg Cys Ser Tyr | |
| 180 185 190 | |
| GGT GCA GAC AGC ACT CTT GGT CCT GTC AAT TTG TCT GCG GAA GGA CCC | 624 |
| Gly Ala Asp Ser Thr Leu Gly Pro Val Asn Leu Ser Ala Glu Gly Pro | |
| 195 200 205 | |
| ACT ACA ATG ACC CTC GTG TGC GGG AAA GAT GGA GTC AAA GTT CCT CAA | 672 |
| Thr Thr Met Thr Leu Val Cys Gly Lys Asp Gly Val Lys Val Pro Gln | |
| 210 215 220 | |
| GAC AAC AAT CAG TAC TGT TCC GGG ACG ACG CTG ACT GGT TGC AAC GAG | 720 |
| Asp Asn Asn Gln Tyr Cys Ser Gly Thr Thr Leu Thr Gly Cys Asn Glu | |
| 225 230 235 240 | |
| AAA TCG TTC AAA GAT ATT TTG CCA AAA TTA ACT GAG AAC CCG TGG CAC | 768 |
| Lys Ser Phe Lys Asp Ile Leu Pro Lys Leu Thr Glu Asn Pro Trp Gln | |
| 245 250 255 | |
| GGT AAC GCT TCG AGT GAT AAG GGT GCC ACG CTA ACG ATC AAG AAG GAA | 816 |
| Gly Asn Ala Ser Ser Asp Lys Gly Ala Thr Leu Thr Ile Lys Lys Glu | |
| 260 265 270 | |
| GCA TTT CCA GCC GAG TCA AAA AGC GTC ATT ATT GGA TGC ACA GGG GGA | 864 |
| Ala Phe Pro Ala Glu Ser Lys Ser Val Ile Ile Gly Cys Thr Gly Gly | |
| 275 280 285 | |
| TCG CCT GAG AAG CAT CAC TGT ACC GTG AAA CTG GAG TTT GCC GGG GCT | 912 |
| Ser Pro Glu Lys His His Cys Thr Val Lys Leu Glu Phe Ala Gly Ala | |
| 290 295 300 | |
| GCA GGG TCA GCA AAA TCG GCT GCG GGA ACA GCC AGT CAC GTT TCC ATT | 960 |
| Ala Gly Ser Ala Lys Ser Ala Ala Gly Thr Ala Ser His Val Ser Ile | |
| 305 310 315 320 | |
| TTT GCC ATG GTG ATC GGA CTT ATT GGC TCT ATC GCA GCT TGT GTC GCG | 1008 |
| Phe Ala Met Val Ile Gly Leu Ile Gly Ser Ile Ala Ala Cys Val Ala | |
| 325 330 335 | |
| TG | 1011 |

Another aspect of the present invention includes novel nucleic acid sequences that encode modified P30 antigens that are functionally equivalent to the natural P30 protein; that is, the modified antigens are capable of eliciting an immune response against T. gondii that protects the animal from toxoplasmosis. These nucleic acid sequences include, but are not limited to, modified nucleic acid sequences that encode P30 antigens from which amino acids possessing potentially troublesome hydrophobic groups (e.g., at the amino and/or carboxyl termini, as necessary) have been removed, P30 antigens which can be secreted from the cells that produce them, and P30 antigens that are able to attach to the outer membranes of the cells that produce them.

When an alphavirus, such as a recombinant virus particle vaccine of the present invention, infects a cell, the virus takes over the host transcription and translation machinery but does not usually kill the cell. Thus, for protective proteins that are often protective only when outside the host cell (e.g., immunogens), it is preferred for such protective proteins to be secreted from the infected cell into a bodily fluid such as the bloodstream, to become attached to the outer membrane of the infected cell, or to be released from infected cells upon cell death.

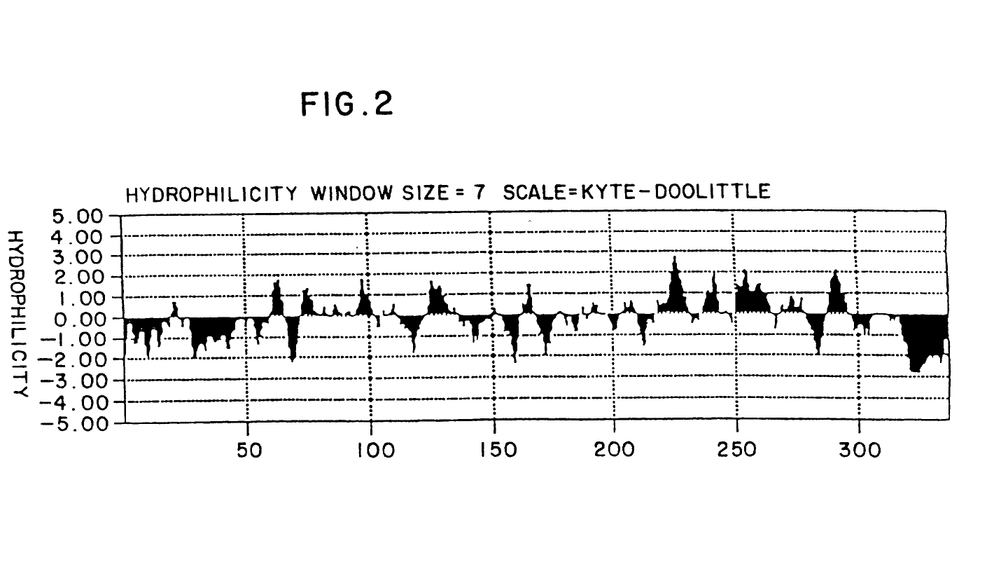

In addition, the amino and carboxyl termini of natural T. gondii P30 antigens are hydrophobic, particularly in the regions spanning amino acid residues from about 1 through about 45 at the amino terminus and spanning amino acid residues from about 309 through about 336 at the carboxyl terminus (assuming that the methionine at position 1 corresponds to the first amino acid of the primary translation product shown in Table 1 and as shown in the hydrophilicity plot in FIG. 2). While not being bound by theory, it is believed that these hydrophobic terminal residues of P30 antigen can lead to protein insolubility problems as well as to an inability to efficiently secrete P30 antigen from the cell in which it is produced. As such, a particularly preferred nucleic acid sequence of the present invention is one in which a nucleic acid encoding a signal segment (i.e., a signal segment nucleic acid) is joined to a nucleic acid sequence encoding a modified T. gondii P30 antigen from which hydrophobic amino and carboxyl terminal residues have been removed in such a manner as to effectively direct secretion of the encoded protective protein from the cell infected by the recombinant virus particle vaccine.

In accordance with the present invention, a signal segment is a peptide that, when joined to a protein in an appropriate manner, directs secretion of that protein from the cell in which the protein was produced. Signal segments, also referred to as signal sequence peptides, usually range in size from about 15 to about 30 amino acids, and are thought to initiate the transport of a protein across the membrane as an early step in the secretion process. In order to direct secretion of a protective protein of the present invention, the signal segment is joined to the amino terminus of the protective protein to be secreted in such a manner that the signal segment is capable of promoting secretion of the protective protein from the recombinant cell in which the protein is produced (e.g., the signal segment is placed “in-frame” with the protective protein). Such a protein is encoded by a signal segment nucleic acid (encoding a signal segment) ligated to a nucleic acid sequence encoding the protective protein in such a manner that the protein produced by the ligated nucleic acid sequences is capable of being secreted.

A suitable signal segment for use in the present invention includes any signal segment capable of directing the secretion of a protein from a cell transfected by a recombinant molecule of the present invention. A nucleic acid encoding a signal segment can be produced by isolating such a nucleic acid from a gene encoding a secreted protein or by chemically synthesizing such a nucleic acid. Preferred signal segments include, but are not limited to, tissue plasminogen activator (t-PA), interferon, interleukin, growth hormone, histocompatibility and viral signal segments. More preferred signal segments include human t-PA, human interferon-α, mouse interleukin-3 and human major histocompatibility complex signal segments. A particularly preferred signal segment is a human t-PA signal segment, preferably including the initial about 23 amino acids of the t-PA primary translation product.

Another preferred nucleic acid sequence of the present invention is one in which a signal segment nucleic acid is substituted for the nucleic acid segment encoding the amino terminal hydrophobic residues of P30 and in which a nucleic acid segment encoding a “hook” or “anchor” is substituted for the nucleic acid segment encoding the carboxyl terminal hydrophobic residues of P30 in order to cause the functionally equivalent protective protein to be attached to the outer membrane of the cell that produces it. Suitable “hooks” include the “hook” carboxyl termini of Class II proteins, such as immunoglobulins.

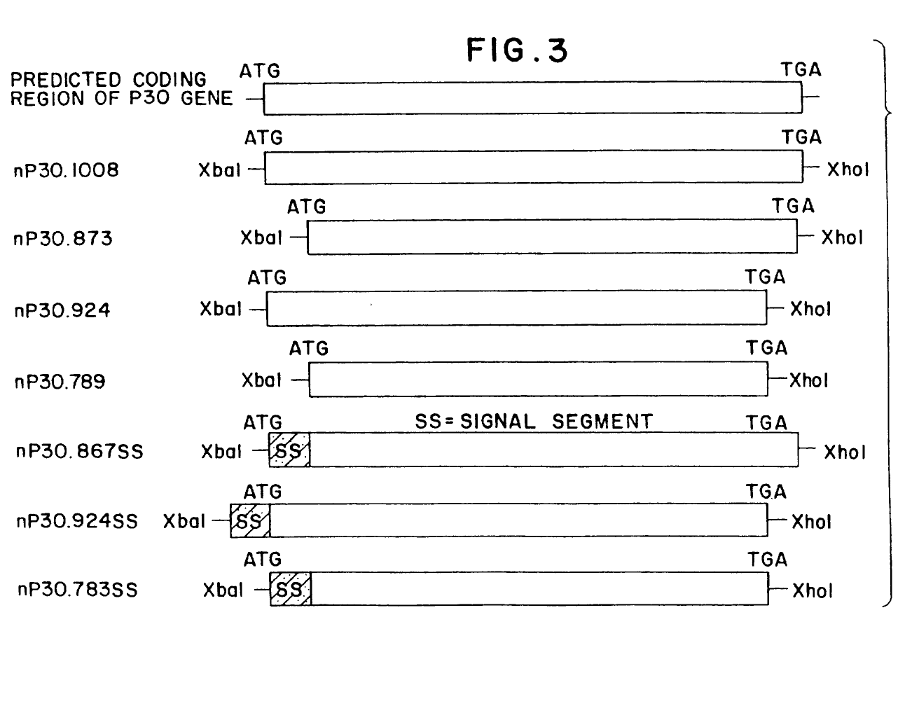

Particularly preferred nucleic acid sequences of the present invention include nP30.1008 which encodes P30 antigen P30.336, corresponding to the amino acid sequence shown encoded in Table 1; nP30.873 which encodes P30.291; nP30.924 which encodes P30.308; nP30.789 which encodes P30.263; nP30.771 which encodes P30.257; nP30.867SS which encodes P30.289SS; nP30.924SS which encodes P30.308SS; nP30.783SS which encodes P30.261SS; and nP30.771SS which encodes P30.257SS. Some of these nucleic acid sequences are depicted in FIG. 3. Note that although the nucleic acid sequences diagrammed in FIG. 3 are flanked by XbaI and XhoI restriction endonuclease sites, such nucleic acid sequences can be flanked by a variety of restriction enzyme sites to allow easy insertion of the sequences into a variety of expression and cloning vectors.

P30.291 spans amino acids from about 46 through about 336 of P30, as numbered in the deduced amino acid sequence of Table 1, and as such, lacks amino terminal hydrophobic residues. P30.308 spans amino acids from about 1 through about 308, as numbered in the deduced amino acid sequence of Table 1, and as such, lacks carboxyl terminal hydrophobic residues. P30.263 spans amino acids from about 46 through about 308, as numbered in the deduced amino acid sequence of Table 1, and as such, lacks both amino and carboxyl terminal hydrophobic residues. P30.257 spans amino acids from about 49 through about 305, as numbered in the deduced amino acid sequence of Table 1, and as such, lacks P30 amino and carboxyl terminal hydrophobic residues. Prior to secretion, P30.289SS contains a t-PA signal sequence of about 23 amino acids joined to the amino terminus of a modified P30 protein that spans amino acids from about 48 through about 336 of P30, as numbered in the deduced amino acid sequence of Table 1, and as such, lacks P30 amino terminal hydrophobic residues. (Note that Burg et al., ibid., have predicted that T. gondii P30 is cleaved between amino acids 47 and 48 during maturation.) Prior to secretion, P30.308SS contains a t-PA signal sequence of about 23 amino acids joined to the amino terminus of a modified P30 protein that spans amino acids from about 1 through about 308 of P30, as numbered in the deduced amino acid sequence of Table 1, and as such, lacks P30 carboxyl terminal hydrophobic residues. Prior to secretion, P30.261SS contains a t-PA signal sequence of about 23 amino acids joined to the amino terminus of a modified P30 protein that spans amino acids from about 48 through about 308 of P30, as numbered in the deduced amino acid sequence of Table 1, and as such, lacks P30 amino and carboxyl terminal hydrophobic residues. Prior to secretion, P30.257SS contains a t-PA signal sequence of about 23 amino acids joined to the amino terminus of a modified P30 protein that spans amino acids from about 49 through about 305 of P30, as numbered in the deduced amino acid sequence of Table 1, and as such, -lacks P30 amino and carboxyl terminal hydrophobic residues. A detailed description of certain preferred nucleic acid sequences of the present invention is presented in the Examples section.

A nucleic acid sequence of the present invention can encode a compound capable of protecting an animal from a disease, including related diseases that are sufficiently similar that a protective compound targeted toward the given disease is also efficacious against related diseases. For example, a nucleic acid sequence of the present invention can encode a compound capable of protecting an animal from disease by one type of parasite, including related parasites that are sufficiently similar to a given parasite that a compound targeted toward the given parasite is also efficacious against related parasites. Another embodiment is a nucleic acid sequence that encodes a multivalent protective compound that is targeted against more than one disease. As such it is possible to protect an animal against more than one (i.e., a combination of) disease. Such a nucleic acid sequence is produced by joining at least two nucleic acid sequences together in such a manner that the resulting sequence is expressed as a multivalent protective compound containing at least two protective compounds, or portions thereof, capable of protecting animals from diseases caused, for example, by at least one infectious agent. Examples of multivalent protective compounds include, but are not limited to, a T. gondii P30 antigen joined to a heartworm antigen, a T. gondii P30 antigen joined -to an antigenic foot and mouth viral protein (e.g., VPl), a T. gondii P30 antigen joined to a hepatitis viral protein, a T. gondii P30 antigen joined to an antigenic human immunodeficiency viral protein and a T. gondii P30 antigen joined to an antigenic feline immunodeficiency viral protein.

As heretofore stated, the present invention includes recombinant molecules that can be either packaging-competent or packaging-defective. In accordance with the present invention, recombinant virus particle vaccines include packaging-defective recombinant molecules. In contrast, both packaging-defective and packaging-competent recombinant molecules can be used in the production of protective compounds in vitro, as described in more detail below. As such, the present invention includes alphavirus expression vectors (and recombinant molecules including such vectors) that can be either packaging-defective or packaging-competent.

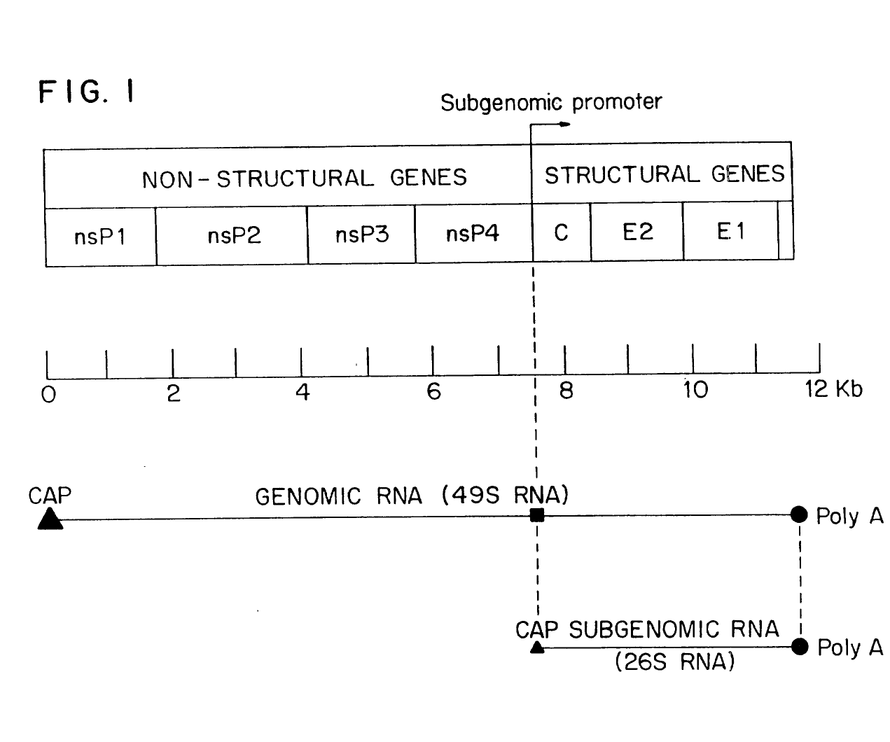

Alphaviruses are RNA viruses with a positive polarity RNA genome of about 12,000 nucleotides in length which sediments at about 49S; see FIG. 1. The 5′ two-thirds of the 49S RNA encodes alphavirus nonstructural polypeptides (e.g., nsP1, nsP2, nsP3 and nsP4, as denoted, for example, for Sindbis virus) required for replication and transcription. Replication of the 49S RNA results in a full-length negative polarity copy which serves both as a template for new genomic RNA and as a template for transcription of a 26S subgenomic RNA molecule corresponding to the 3′ third of the genome, which contains the genes for alphavirus structural polypeptides (e.g., capsid polypeptide C, envelope glycopeptides El and E2, E3 and 6K, as denoted, for example for Sindbis virus).

An alphavirus expression vector of the present invention can be either a DNA or RNA vector that contains alphavirus sequences that alone, or in concert with other sequences, are capable of directing expression of at least one protective compound of the present invention. The phrase “capable of directing expression of at least one protective compound” refers to the ability of an alphavirus expression vector of the present invention, when placed in an appropriate host cell, to use the cellular machinery, as well as its own regulatory control regions and/or encoded enzymes, to effect transcription of genes present on the recombinant molecule, including the nucleic acid sequence to which the expression vector is operatively linked. Use of alphavirus expression vectors of the present invention is advantageous because such vectors are capable of directing the production of large amounts of biologically active compounds in the cytoplasm of cells transfected by such vectors or infected by recombinant virus particles including such vectors.

Packaging-competent alphavirus expression vectors can include genes encoding alphavirus nonstructural polypeptides and all of the alphavirus structural polypeptides (e.g., capsid polypeptide C and envelope glycopolypeptides E1 and E2). Alternatively, one or more of the structural genes can be encoded by the animal cell into which a recombinant molecule is transfected. (Such an animal cell can be produced, for example, by introducing such structural genes into the cell in a manner such that the genes are integrated into the cellular genome).

Packaging-defective alphavirus expression vectors include alphavirus nonstructural genes but lack the ability to produce one or more functional structural polypeptides. Such inability may be due to lack of a gene encoding the structural polypeptide and/or to at least one mutation in a structural gene such that a functional structural polypeptide cannot be produced. Such a packaging-defective recombinant molecule is able to be transcribed within a cell but cannot be packaged into an infectious virus unless a helper virus is present. That is, the phrases “packaging-defective” and “not capable of effecting packaging of said recombinant molecule” each refers to the inability of a packaging-defective alphavirus expression vector alone to accomplish packaging of a packaging-defective recombinant molecule into a virus since such an alphavirus expression vector does not contain a complete copy of the genes that encode the structural polypeptides that make up the viral coat. Preferred packaging-defective alphavirus expression vectors of the present invention retain the site required for packaging within the nonstructural polypeptide nsP1 but lack the ability to produce one or more functional alphavirus structural polypeptides required to effect packaging of the recombinant molecule. The size of packaging-defective alphavirus expression vectors of the present invention is a function of the size of the recombinant molecules that contain the vectors since the recombinant molecules must be of a size appropriate to be packaged into viral particle vaccines according to the method described below.

An alphavirus expression vector of the present invention is preferably able to direct replication of a recombinant molecule of the present invention, meaning that when the recombinant molecule is placed in an appropriate host cell, the alphavirus expression vector is able to use the host cell machinery, as well as its own regulatory control regions and/or encoded enzymes, to effect replication of the recombinant molecule. In a preferred embodiment, the alphavirus expression vector includes genes encoding each of the alphavirus nonstructural polypeptides (e.g., nonstructural polypeptides nsP1, nsP2, nsP3, and nsP4), or functional equivalents thereof, and other regulatory control regions required for transcription and replication of the recombinant molecule. A preferred packaging-defective recombinant molecule of the present invention contains genes encoding each of the alphavirus nonstructural polypeptides (e.g., nonstructural polypeptides nsP1, nsP2, nsP3, and nsP4), or functional equivalents thereof, and other signals required for transcription and replication of the recombinant molecule, but lacks the ability to produce one or more functional structural polypeptides (e.g., capsid polypeptide C or envelope glycopolypeptides-E1 or E2). Such a recombinant molecule is able to be transcribed and replicated within a cell but cannot be packaged into an infectious virus unless a helper virus is present.

An alphavirus expression vector of the present invention can comprise any alphavirus expression vector and can be a hybrid between at least two alphavirus vectors. As used herein, a hybrid alphavirus expression vector refers to an expression vector that contains different regions from different alphavirus genomes that in combination (i.e., ligated together) have the properties of an alphavirus expression vector of the present invention. Preferred alphavirus expression vectors include Sindbis virus expression vectors, Semliki Forest virus expression vectors, Ross River virus expression vectors, Venezuela equine encephalitis virus and hybrids thereof. Sindbis virus expression vectors are particularly preferred alphavirus expression vectors despite a statement by Liljestrom et al., ibid., that technical difficulties (e.g., low transfection rates) have precluded wide spread use of Sindbis virus vectors. According to the present invention, either lipofection or electroporation permits straightforward manipulation of Sindbis virus vectors.

Sindbis virus vectors are particularly preferred because, unlike a number of other alphaviruses, Sindbis virus is not known to be associated with human disease. In addition, Sindbis virus has a wide host range. For example, Sindbis virus can infect mammalian, avian, insect, amphibian, and reptilian cells. Sindbis virus can also infect a number of cell types, including, but not limited to, Chinese hamster ovary cells, baby hamster kidney cells, quail (e.g., QT-6) cells, chicken embryo fibroblasts, human tumor cells, mosquito and Drosophila cells. Sindbis virus can also be transmitted to vertebrate hosts, such as birds or mammals, by mosquitos.

Sindbis virus gene expression, which occurs in the cytoplasm of the cell, is quite efficient, rapid, and can be modulated. For example, Xiong et al., ibid., reported the production of up to 1×10⁸ molecules of chloramphenicol acetyltransferase (CAT) per cell transfected with Sindbis virus expression vectors operatively linked to the CAT gene, when the cell was cultured for about 20 hr. Xiong et al. also reported that use of a replication temperature sensitive Sindbis virus vector led to modulated expression of CAT.

An alphavirus expression vector of the present invention preferably contains an alphavirus subgenomic promoter which, in natural alphavirus isolates, controls expression of viral structural polypeptide genes. In a preferred embodiment, expression of a nucleic acid sequence of the present invention is operatively joined to such an alphavirus subgenomic promoter. In one embodiment, at least one of the structural polypeptide genes of a natural alphavirus vector is deleted and a nucleic acid sequence of the present invention is joined to the vector such that expression of the nucleic acid sequence is placed under the control of alphavirus subgenomic promoter. In another embodiment, a nucleic acid sequence encoding the protective compound is operatively linked to the subgenomic promoter in combination with the structural genes. In yet another embodiment, the recombinant molecule can include a subgenomic promoter operatively linked to alphavirus structural genes and a second subgenomic promoter operatively linked to a nucleic acid sequence encoding the protective compound. Use of alphavirus subgenomic promoters are advantageous because they lead to high levels of protein production in relatively short periods of time. However, it should be appreciated that other suitable promoters may also be used to control expression of a protective protein of the present invention.

Suitable alphavirus subgenomic promoters of the present invention include any alphavirus subgenomic promoter, including hybrids thereof. A hybrid alphavirus subgenomic promoter is a promoter in which different regions thereof are derived from different alphavirus subgenomic promoters. Preferred subgenomic promoters include subgenomic promoters of Sindbis virus, Semliki Forest virus, Ross River virus, Middleburg virus, O'Nyong-nyong virus, Eastern equine encephalitis virus, Western equine encephalitis virus, Venezuelan equine encephalitis virus, and hybrids thereof. More preferred alphavirus subgenomic promoters include Sindbis virus subgenomic promoters, Semliki Forest virus subgenomic promoters, Ross River virus subgenomic promoters, Venezuelan equine encephalitis virus and hybrids thereof, with Sindbis virus subgenomic promoters being even more preferred.

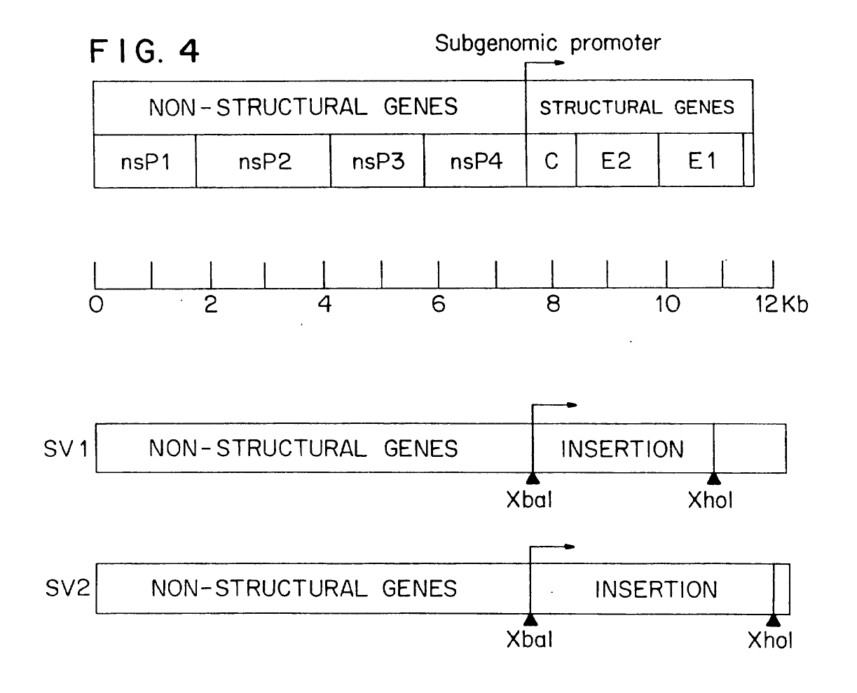

A recombinant virus particle vaccine of the present invention preferably includes a packaging-defective alphavirus vector in which at least one of the structural polypeptide genes of a natural alphavirus vector is deleted and a nucleic acid sequence of the present invention is joined to the vector such that expression of the nucleic acid sequence is under the control of alphavirus subgenomic promoter. Particularly preferred packaging-defective Sindbis virus expression vectors include SV1 and SV2, which are depicted in FIG. 4. SV1 contains all the genes encoding nonstructural Sindbis virus polypeptides, the subgenomic promoter plus about 14 nucleotides downstream from the subgenomic RNA initiation site, and about 616 nucleotides at the 3′ end of the Sindbis viral genome plus the poly(A) tail. SV2 is similar to SV1 except that SV2 contains only about 62 nucleotides of the 3′ end of the Sindbis viral genome, thereby permitting the insertion of a larger nucleic acid sequence. Each of these vectors has a site for insertion of a nucleic acid sequence of the present invention as indicated.

In accordance with one embodiment of the present invention, a recombinant molecule can be produced by (a) isolating a nucleic acid sequence that encodes a protective protein, (b) isolating an alphavirus expression vector capable of directing transcription of a recombinant molecule of which the vector is a part, and (c) operatively linking the nucleic acid sequence to the expression vector to obtain a recombinant molecule in which expression of the nucleic acid sequence is controlled by the expression vector. Preferably, the alphavirus expression vector is also capable of directing replication of the recombinant molecule.

Techniques for isolating nucleic acid sequences and expression vectors and for operatively linking a coding sequence to an expression vector are described in detail in Sambrook et al., Ibid. Since it is technically difficult to perform recombinant techniques on RNA viruses, the RNA alphavirus vectors of the present invention are preferably converted into double-stranded cDNA copies using standard techniques. After genetic manipulations, such as the insertion of a nucleic acid sequence of the present invention into an alphavirus expression vector, the resultant DNA recombinant molecule can be transcribed into an RNA recombinant molecule, for example, by the following method: The DNA recombinant molecule is inserted into a plasmid containing an RNA polymerase promoter and is transcribed in vitro in the presence of an appropriate RNA polymerase and other reagents to effect transcription. These techniques are described in greater detail in Sambrook et al., ibid., and Xiong et al., ibid. Suitable RNA polymerase promoters include, but are not limited, to bacteriophage SP6, T7 and T3 promoters. A preferred RNA polymerase promoter is the bacteriophage SP6 promoter (e.g., Melton et al., pp. 7035-7056, 1984, Nucleic Acids Research, Vol. 12). The DNA recombinant molecule to be transcribed is preferably a linear or supercoiled molecule, with linear being more preferred.

Preferred recombinant molecules of the present invention include alphavirus expression vectors operatively linked to preferred nucleic acid sequences of the present invention. A recombinant molecule of the present invention can include one or more nucleic acid sequences operatively linked to one or more transcription control sequences (e.g., alphavirus subgenomic promoters). A preferred recombinant molecule of the present invention includes a Sindbis virus expression vector and at least one nucleic acid sequence operatively linked to a Sindbis virus subgenomic promoter, the nucleic acid sequence encoding a T. gondii P30 antigen, a D . immitis P39 antigen, a D. immitis P22L antigen, a D . immitis P22U antigen, a D. immitis P20.5 antigen, a D . immitis P4 antigen, a D. immitis Di22 antigen (such as the Di22.RA antigen described in the Examples and/or an L3 and/or L4 D. immitis protease.

Recombinant virus particle vaccines of the present invention include packaging-defective recombinant molecules that can be produced as heretofore disclosed using packaging-defective alphavirus expression vectors. For example, a packaging-defective recombinant molecule can be produced by (a) isolating a nucleic acid sequence that encodes a natural or functionally equivalent T. gondii P30 protein capable of protecting an animal from toxoplasmosis; (b) isolating a Sindbis virus expression vector that contains a subgenomic promoter and that encodes each of the Sindbis virus nonstructural polypeptides, but that is unable to encode at least one functional Sindbis virus structural polypeptide; and (c) operatively linking the P30 sequence to the subgenomic promoter so that expression of P30 is controlled by the subgenomic promoter. In one embodiment, at least one of the genes encoding Sindbis virus structural polypeptides C, E1, or E2 is replaced by the sequence encoding a P30 antigen.

Preferred packaging-defective recombinant molecules of the present invention include alphavirus expression vectors operatively linked to preferred nucleic acid sequences of the present invention. Particularly preferred recombinant molecules of the present invention include SV1: nP30.1008, SV1: nP30.924, SV1:nP30.873, SV1: nP30.789, SV1: nP30.771, SV1: nP30.924SS, SV1: nP30.867SS, SV1: nP30.783SS, SV1: nP30.771SS, SV2:nP30.1008, SV2:nP30.924, SV2:nP30.873, SV2:nP30.789, SV2:nP30.771, SV2:nP30.924SS, SV2:nP30.867SS, SV2:nP30.783SS, and SV2:nP30.771SS. The name of each of these particularly preferred recombinant molecules indicates the alphavirus expression vector (e.g., SV1) to which the nucleic acid sequence (e.g., nP30.1008) is operatively linked (:).

In accordance with the present invention, a recombinant virus particle vaccine can be produced by a method which includes the steps of (a) co-transfecting a host cell with a packaging-defective recombinant molecule and an alphavirus packaging vector; (b) culturing the transfected cell in an effective medium to produce a recombinant virus particle; (c) recovering the particle; and (d) formulating a vaccine therefrom. Preferably, the vaccine comprises a packaging-defective recombinant molecule packaged in a viral coat that includes alphavirus structural polypeptides such as capsid polypeptide (C) and two envelope glycopolypeptides (E1 and E2). While not being bound by theory, it is believed that the recombinant molecule complexes with the capsid polypeptide to form an intracellular icosahedral nucleocapsid which interacts with the cytoplasmic domains of the transmembrane envelope polypeptides E1 and E2, resulting in the budding of the virus vaccine at the plasma membrane.

Since the packaging-defective recombinant molecules of the present invention do not themselves encode all the polypeptides required for packaging, the components of the viral coat can be provided by co-transfecting a host cell with both a packaging-defective recombinant molecule and an alphavirus packaging vector which acts as a helper virus to package the packaging-defective recombinant molecule. As used herein, “an alphavirus packaging vector” is an alphavirus-based vector that contains the genes that encode the structural polypeptides required for packaging of a packaging-defective recombinant molecule of the present invention into a virus particle. The packaging vector also contains sequences corresponding to the 5′ and 3′ ends of alphavirus RNA molecules which are important in transcription and replication. However, the alphavirus packaging vector is unable to direct its own packaging (i.e., self-package) because it lacks the site located within the nsP1 gene thought to be necessary for packaging to occur. As such, packaging vectors of the, present invention are much more useful for packaging packaging-defective recombinant molecules than are viral genomes containing the entire alphavirus RNA molecule since co-transfection of a host cell with a packaging-defective recombinant molecule and a packaging vector of the present invention results in the desired recombinant virus particle vaccine but does not lead to the production of infectious alphaviruses (i.e., alphaviruses which are able to replicate and package themselves).

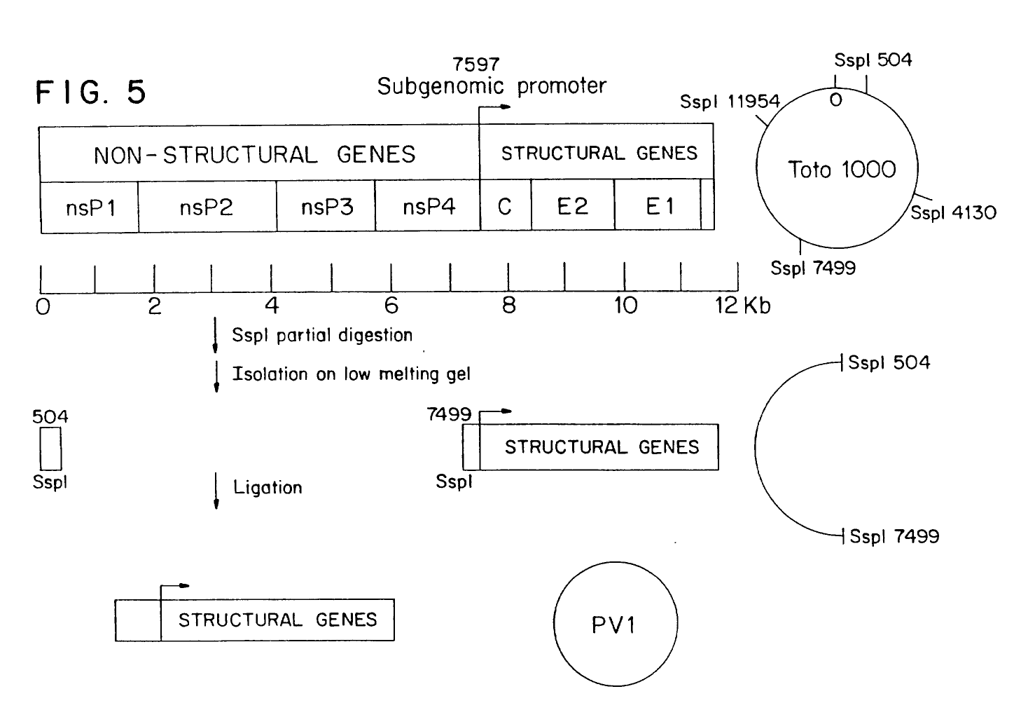

Suitable alphavirus packaging vectors include Sindbis virus packaging vectors, Semliki Forest virus packaging vectors, Ross River virus packaging vectors, Venezuelan equine encephalitis virus packaging vectors and hybrids thereof. Sindbis virus packaging vectors are preferred, particularly those that contain a minimal amount of genetic information to effect packaging. While not being bound by theory, it is believed that smaller packaging vectors are better because they are more efficient and more RNA can be produced per unit time. Particularly preferred packaging vectors are Sindbis virus packaging vectors that contain the structural polypeptide genes under the control of the subgenomic promoter and also contain replication and transcription signals at the 5′ and 3′ ends of Sindbis viral RNA. A particularly preferred Sindbis virus packaging vector is PV1, the production of which is depicted in FIG. 5.

According to the present invention, a recombinant virus particle vaccine can also be produced by (a) introducing, by transfection, a packaging-defective recombinant molecule into a host cell that already contains genes integrated into its chromosomal DNA and/or on extrachromosomal vectors that encode the structural polypeptides required to effect packaging of the packaging-defective recombinant molecule (i.e., a host cell that is capable of packaging the packaging-defective recombinant molecule into a virus particle); (b) culturing the transfected cell in an effective medium to produce the virus particle; (c) recovering the virus particle; and (d) formulating a vaccine therefrom. Preferred genes are structural polypeptide genes of Sindbis virus, Semliki Forest virus, Ross River virus, Venezuelan equine encephalitis virus, with the structural polypeptide genes of Sindbis virus being more preferred. For example, Chinese hamster ovary cells containing genes encoding alphavirus structural polypeptides C, E1, E2, E3, and 6K, or functional equivalents thereof of said polypeptides can be used as host cells.

A number of host cells are suitable for recombinant virus particle vaccine production since alphaviruses have such wide host ranges. Suitable host cells include, but are not limited to, mammalian, insect, avian, reptilian, amphibian, and some insect (e.g., mosquito and Drosophila) cells. Preferred host cells include mammalian, insect, and avian cells. More preferred host cells include Chinese hamster ovary cells, baby hamster kidney cells, chicken embryonic fibroblasts, and mosquitos.

As used herein, transfection includes any means for introducing a nucleic acid sequence, expression vector, recombinant molecule, or packaging vector, into a host cell, including, but not limited to transformation, electroporation, microinjection, lipofection, adsorption, and protoplast fusion. Preferred transfection techniques are lipofection and electroporation.

After transfection, transfected cells are cultured in an effective medium, using techniques such as those described in Xiong et al., ibid. As used herein, an effective medium refers to any medium in which the transfected cells can produce recombinant virus particle vaccines. An effective medium is typically an aqueous medium comprising assimilable carbohydrate, nitrogen and phosphate sources, as well as appropriate salts, minerals, metals and other nutrients, such as vitamins, growth factors and hormones. Culturing is carried out at a temperature, pH and oxygen content appropriate for the transfected cell. , Such culturing conditions are well within the expertise of one of ordinary skill in the art. Examples of preferred effective media are included in the Examples section.

Recombinant virus particles can be recovered from the cultured transfected cells using a combination of standard techniques such as, but not limited to, affinity chromatography, ion exchange chromatography, filtration, and hydrophobic interaction chromatography. A preferred recovery technique is Matrex® Cellufine™ Sulfate Media & Virus Recovery System, available from Amicon Inc., Danvers, Mass.

Due to the nature of the recombinant molecules and packaging vectors of the present invention, essentially no infectious virus is formed (i.e., the probability of forming infectious virus is less than about 1×10⁻⁶), thus simplifying recovery of recombinant virus particle vaccines. Sindbis virus recombinant molecules and packaging vectors are particularly preferred since, even if a small amount of infectious Sindbis virus is produced, the virus is safe.

Preferably, a recombinant virus particle of the present invention is recovered in “substantially pure” form. As used herein, “substantially pure” refers to a purity that allows for the effective use of the recombinant virus particle as a vaccine without substantial negative side effects. One embodiment of a substantially pure virus particle is a cell lysate containing the virus particle that generates substantially no side effects when administered to an animal in an effective amount to protect the animal from disease. It is within the scope of the present invention to recover recombinant virus particles having a purity of up to and including about 99 percent.

A recombinant virus particle vaccine of the present invention, when administered to an animal in an effective amount, infects the cells of the animal (in a manner essentially harmless to the animal) and directs the production of a protective compound able to protect the animal from an infectious or metabolic disease. Preferred recombinant virus particle vaccines are those that protect animals from infection by the infectious agents heretofore disclosed. Particularly preferred vaccines are those that protect animals from toxoplasmosis and/or heartworm.

A preferred recombinant virus particle vaccine of the present invention includes a packaging-defective recombinant molecule packaged in a Sindbis virus coat in which the recombinant molecule contains a nucleic acid sequence encoding a T. gondii and/or a D. immitis antigen capable of protecting an animal from toxoplasmosis and/or heartworm operatively linked to a Sindbis virus expression vector. More preferred recombinant virus particle vaccines (VPVs) include: VPV SV1: nP30.1008, VPV SV1: nP30.924, VPV SV1:nP30.873, VPV SV1:nP30.789, VPV SV1:nP30.771, VPV SV1:nP30.924SS, VPV SV1:nP30.867SS, VPV SV1:nP30.783SS, VPV SV1:nP30.771SS, VPV SV2:nP30.1008, VPV SV2:nP30.924, VPV SV2:nP30.873, VPV SV2:nP30.789, VPv SV2:nP30.771, VPV SV2:nP30.924SS, VPV SV2:nP30.867SS, VPV SV2:nP30.783SS, and VPV SV2:nP30.771SS. Each of these vaccines includes the designated recombinant molecule packaged in a Sindbis virus coat. For example, vaccine VPV SV1:nP30.1008 includes recombinant molecule SV1:nP30.1008 packaged in a Sindbis virus coat; recombinant molecule SV1:nP30.1008 contains a nucleic acid sequence that encodes a T. gondii P30 antigen of 336 amino acids that is operatively linked to alphavirus expression vector SV1.

Recombinant virus particle vaccines of the present invention can be used to protect animals from a variety of diseases, including infectious and metabolic diseases. When administered to an animal, the recombinant virus particle vaccine infects cells within the immunized animal and directs the production of a protective protein or RNA species that is capable of protecting an animal from disease. For example, a T. gondii antigen will protect an animal from toxoplasmosis, a feline or human immunodeficiency virus (FIV or HIV, respectively) antigen will protect an animal from FIV or HIV infection, a D. immitis antigen will protect an animal from heartworm, a coccidia antigen will protect an animal from coccidiosis, a Plasmodium falciparum antigen will protect an animal from malaria, a Cryptosporidium antigen will protect an animal from enteric disease, an Encephalitozoon cuniculi antigen will protect an animal from encephalitozoonosis, and a Pneumocystis antigen will protect an animal from pneumonia.

Vaccines of the present invention can be administered to any animal, preferably to mammals, birds and insects, and more preferably to humans, cats, dogs, sheep, pigs, cattle, horses, poultry, ferrets, and other pets and/or economic food animals. Particularly preferred animals to protect include humans, cats and dogs. A preferred vaccine is one that, when administered to an animal, is preferably able to elicit (i.e., stimulate) the production of very high antibody titers as well as a high-level cellular immune response to the protective protein encoded by the nucleic acid sequence. Administration of a vaccine containing multiple nucleic acid sequences targeting multiple infectious agents can protect the vaccinated animal from those multiple infectious diseases.

Vaccines can be formulated in an aqueous balanced salt solution, or other excipients as disclosed below, that the animal to be vaccinated can tolerate. In one embodiment of the present invention, the vaccine can also include an immunopotentiator, such as an adjuvant or a carrier. One advantage of live virus-based vaccines, such as the recombinant virus particle vaccines of the present invention, is that adjuvants and carriers are not required to produce an efficacious vaccine. However, it should be noted that use of immunopotentiators is not precluded by the present invention.

Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark).

Carriers are typically compounds that increase the half-life of a vaccine in a vaccinated animal. Suitable carriers include, but are not limited to, polymeric controlled release formulations, biodegradable implants, liposomes, bacteria, other viruses, oils, esters, and glycols.

In order to protect animals from a disease, a recombinant virus particle vaccine of the present invention is administered in an effective amount, wherein an “effective amount” is an amount that allows the animal to produce sufficient protective protein or RNA species to protect itself from the disease. For example, when the protective protein is a T. gondii antigen, the recombinant virus particle vaccine is administered according to a protocol that results in the animal producing a sufficient immune response to protect itself from toxoplasmosis. The administration protocol includes individual dose size, number of doses, frequency of dose administration, and mode of administration. A suitable single dose of the vaccine is a dose that is capable of protecting an animal from a disease when administered one or more times over a suitable time period. A preferred single dose of the vaccine is from about 1×10⁴ to about 1×10⁶ virus plaque forming units (pfu) per kilogram (kg) body weight of the animal. Booster vaccinations can be administered from about 2 weeks to several years after the original vaccination. Preferably booster vaccinations are administered when the immune response of the animal becomes insufficient to protect the animal from disease. A preferred administration schedule is one in which from about 1×10⁴ to about 1×10⁶ virus plaque forming units per kilogram (kg) body weight of the animal are administered from about 1 to about 2 times over a time period of from about 12 to about 18 months. Modes of administration can include, but are not limited to, subcutaneous, intradermal, intravenous, nasal, oral, transdermal and intramuscular routes.

The efficacy of a recombinant virus particle vaccine of the present invention to protect an animal from disease can be tested in a variety of ways including, but not limited to, detection of protective protein or RNA species within the vaccinated animal or challenge of the vaccinated animal with an appropriate infectious agent to determine whether the animal is now resistant to the disease caused by such an agent. When the protective protein is an immunogen, it is also possible to determine vaccine efficacy by measuring antibody production by the animal in response to the immunogen (using either the immunogen or corresponding infectious agent as the target) and/or determining the ability of immune response cells (e.g. splenocytes) to respond to the infectious agent at various effector:target ratios.

One method to determine the ability of a nucleic acid of the present invention to encode a protective protein capable of eliciting an immune response against a disease, such as toxoplasmosis, is as follows. A recombinant molecule of the present invention is transfected into a host cell, preferably into a mammalian, insect, or avian cell. The host cell is cultured under conditions that promote production of the protective protein (e.g., a T. gondii P30 antigen), which subsequently can be recovered from the culture. The recovered protective protein is then injected one or more times into an animal, such as a rabbit, in a manner to promote the production of antibodies against the protective protein. Serum from the rabbit is subsequently recovered and tested for its ability to bind to, for example, the recovered protective protein, the corresponding native protective protein, and the corresponding infectious agent, with affinities that suggest that the nucleic acid encodes a suitable immunogen. For example, in the case of a T. gondii P30 antigen, the serum is tested against recovered P30 antigen, native T. gondii P30 antigen, and T. gondii tachyzoite parasites.

In one embodiment of the present invention, a recombinant virus particle vaccine, preferably one encoding a T. gondii antigen, and more preferably VPV SV1:nP30.1008 is administered subcutaneously to an animal, preferably a mammal, one or more times over a time period of from about 2 to about 4 weeks. Vaccine efficacy can be measured, for example, by determining whether the serum of the vaccinated animal contains antibodies that react with either a T. gondii parasite or the T. gondii antigen encoded by the nucleic acid sequence in the vaccine and/or, preferably, by challenging the animal with a dose of T. gondii parasites and determining if the animal develops toxoplasmosis. Protection can be monitored, for example, by mortality or by assaying for brain cysts.

In accordance with one embodiment of the present invention, the efficacy of a recombinant virus particle vaccine of the present invention may be improved by co-administering the recombinant virus particle vaccine with a protective compound encoded by the nucleic acid sequence of the recombinant virus particle vaccine. While not being bound by theory, it is believed that administration of a protective protein in conjunction with a recombinant virus particle capable of producing the protein may boost particularly the antibody titer. The protective protein can be administered prior to, concomitant with, and/or following administration of the recombinant virus particle vaccine. The protective protein can be either native (naturally-occurring), synthetic, or recombinant. The protective protein can be a natural protein or functional equivalent thereof. The protective protein should be substantially pure, meaning that the protein is sufficiently pure to allow for effective use of the protective protein as a vaccine; i.e., the protein does not cause substantial side effects. The protective protein can be joined (i.e., conjugated) to a carrier or other material that enhances the immunogenicity of the protective protein. Suitable protective proteins, as well as preferred methods to produce such proteins, are disclosed below. In one embodiment, a recombinant virus particle vaccine containing a nucleic acid sequence encoding a T. gondii P30 antigen is administered with a sufficiently pure T. gondii P30 antigen, such as, but not limited to, a native P30 antigen (Khan et al., ibid., Bulow et al., ibid.), a recombinant P30 antigen such as P30.336, P30.308, P30.291, P30.289, P30.263, P30.261, P30.257; or a fusion protein between a recombinant P30 antigen and a fusion segment, such as GST-P 30.336, GST-P30.308, GST-P30.291, GST-P30.289, GST-P 30.263, GST-P30.261 or GST-P30.257, which are described in more detail below.

The present invention also includes a rapid and efficient method to produce, in eukaryotic cells, compounds protective against parasitic infection. As such, the present invention includes recombinant molecules that are capable of directing the expression of at least one compound capable of protecting an animal from parasitic disease (i.e., disease caused by a parasite) when the recombinant molecule is transfected into an animal cell (i.e., introduced into an animal cell such that the transfected cell is capable of producing the protective compound). As heretofore stated, a recombinant molecule of the present invention includes at least one nucleic acid sequence encoding such a compound operatively linked to an alphavirus expression vector. Suitable and preferred nucleic acid sequences include those heretofore cited for use in recombinant virus particle vaccines that are capable of protecting an animal from one or more parasitic diseases, including nucleic acid sequences that encode one or more protective proteins, one or more protective RNA species, one or more protective proteins including a signal segment, one or more protective proteins including a fusion segment, and one or more protective proteins including both a signal segment and a fusion segment.

A nucleic acid sequence encoding a fusion protein refers herein to a nucleic acid sequence encoding a protective protein that includes at least one fusion segment attached to the protective moiety of the protective protein. Such a protein can also include a signal segment if secretion of such a protein is desired. Inclusion of a fusion segment in a protective protein can enhance the protective protein's stability during production, storage and/or use. Depending on the segment's characteristics, a fusion segment can also act as an immunopotentiator to enhance the immune response mounted by an animal immunized with a protective protein containing such a fusion segment. Furthermore, a fusion segment can function as a tool to simplify purification of a protective protein, such as to enable purification of the resultant fusion protein using affinity chromatography. A suitable fusion segment can be a domain of any size that has the desired function (e.g., increased stability, increased immunogenicity, and/or purification tool). It is within the scope of the present invention to use one or more (i.e., a combination thereof) fusion segments. Fusion segments can be joined to amino and/or carboxyl termini of the protective portion of the protective protein. The linkage between fusion segments and the protective portions of protective proteins can be susceptible to cleavage in order to enable straight-forward recovery of the protective portions of such protective proteins.

Preferred fusion segments for use in the present invention include a glutathione binding domain, such as Schistosoma japonicum glutathione-S-transferase (GST) or a portion thereof capable of binding to glutathione; a metal binding domain, such as a poly-histidine segment capable of binding to a divalent metal ion; an immunoglobulin binding domain, such as Protein A, Protein G, T cell, B cell, Fc receptor or complement protein antibody-binding domains; and/or a sugar binding domain such as β-galactosidase or maltose binding protein. Particularly preferred protective proteins include a glutathione binding domain, such as the carboxyl terminal region of S. japonicum GST, or a metal binding domain, such as a poly-histidine segment. Particularly preferred nucleic acid sequences of the present invention that encode fusion proteins include nGST-nP 30.1008 which encodes GST-P30.336; nGST-nP30.873 which encodes GST-P30.291; nGST-nP30.924 which encodes GST-P 30.308; nGST-nP30.789 which encodes GST-P30.263; nGST-nP 30.771 which encodes GST-P30.257; nGST-nP30.867SS which encodes GST-P30.289SS; nGST-nP30.924SS which encodes GST-P 30.308SS; nGST-nP30.783SS which encodes GST-P30.261sS; and nGST-nP30.771SS which encodes GST-P30.257SS. The nGST-containing nucleic acid sequences are similar to the nucleic acid sequences depicted in FIG. 3 except that a nucleic acid segment encoding a S. japonicum GST glutathione binding domain has been inserted immediately upstream from the nucleic acid sequence encoding the specified T. gondii P30 antigen. Note that for nucleic acid sequences encoding secretable antigens, the nucleic acid sequence is engineered such that the encoded protein comprises a glutathione binding domain inserted between the signal segment and the specified T. gondii P30 antigen. As such, these nucleic acid sequences encode fusion proteins including GST joined to modified T. gondii P30 antigens. A detailed description of certain of such nucleic acid sequences is presented in the Examples section.

In accordance with the present invention, recombinant molecules for use in the production of protective compounds in eukaryotic cells can be either packaging-competent or packaging-defective. Such recombinant molecules can be produced using methods heretofore disclosed. Packaging-defective recombinant molecules typically have essentially no restrictions with respect to the size of heterologous nucleic acid sequences that can be incorporated. Furthermore, they do not lead to the production of infectious particles. Packaging-competent recombinant vectors, in contrast, can effect self-packaging into viruses, thereby resulting in infection of cells over multiple generations.

Packaging-competent- and packaging-defective recombinant molecules for use in the production of protective compounds in eukaryotic cells contain, respectively, packaging-competent and packaging-defective alphavirus expression vectors as heretofore described. Such alphavirus expression vectors can include hybrid vectors as heretofore described. Nucleic acid sequences encoding protective compounds are preferably operatively linked to alphavirus subgenomic promoters as heretofore described. Preferred vectors and promoters are as heretofore described, with Sindbis expression vectors including Sindbis subgenomic promoters being particularly preferred.

Preferred Sindbis virus expression vectors for use in producing protective compounds in eukaryotic cells include packaging-defective vectors SV1 and SV2, as well as packaging-competent vectors SV3, SV4, SV5 and SV6. SV1 and SV2, depicted in FIG. 4, are described in detail above. SV3 and SV4 each contain all the genes encoding nonstructural Sindbis virus polypeptides, all the genes encoding structural Sindbis virus polypeptides under the control of a Sindbis subgenomic promoter, an additional subgenomic promoter to which a nucleic acid sequence encoding a protective compound can be operatively linked, and about 62 nucleotides at the 3′ end of the Sindbis viral genome plus the poly(A) tail. In SV3, the gene order (5′ to 3′) is nonstructural polypeptide genes, structural polypeptide genes, nucleic acid sequence(s) encoding protective compound(s). In SV4, the gene order is nonstructural polypeptide genes, nucleic acid sequence(s) encoding protective compound(s), structural polypeptide genes. SV5 is similar to SV3 except that a single subgenomic promoter controls expression of both the structural polypeptide genes and the nucleic acid sequence(s) encoding protective compound(s). SV6 is similar to SV4 except that a single subgenomic promoter controls expression of both the nucleic acid sequence(s) encoding protective compound(s) and structural polypeptide genes. Sindbis expression vectors SV1, SV2, SV3, SV4, SV5 and SV6 each has a site for insertion of a nucleic acid sequence of the present invention, the site being flanked by XbaI and XhoI restriction endonuclease sites; see, for example, the depiction of SV1 and SV2 in FIG. 4. It can be appreciated, however, by one skilled in the art that such insertion sites can be flanked by a variety of restriction enzyme sites to allow easy insertion of nucleic acid sequences of the present invention into a variety of expression and cloning vectors.

A preferred recombinant molecule for use in producing protective compounds in eukaryotic cells in accordance with the present invention includes an alphavirus expression vector operatively linked to at least one preferred nucleic acid sequence of the present invention. Such a recombinant molecule can include one or more nucleic acid sequences operatively linked to one or more transcription control sequences (e.g., alphavirus subgenomic promoters). A more preferred recombinant molecule includes a Sindbis virus expression vector and at least one nucleic acid sequence operatively linked to a Sindbis virus subgenomic promoter, the nucleic acid sequence encoding a T. gondii P30 antigen, a D . immitis P39 antigen, a D . immitis P22L antigen, a D. immitis P22U antigen, a D . immitis P20.5 antigen, a D. immitis P4 antigen, a D. immitis Di22 antigen (such as the Di22.RA antigen described in the Examples) and/or an L3 and/or L4 D. immitis protease. Preferably, the antigen also includes a signal segment, such as a t-PA signal segment, capable of promoting secretion of the antigen and/or a fusion segment, such as a glutathione binding domain or metal binding domain. Particularly preferred recombinant molecules of the present invention comprise recombinant molecules in which SV1, SV2, SV3, SV4, SV5 or SV6 is operatively linked to at least one of the following nucleic acid sequences: nP30.1008, nP30.924, nP30.873, nP30.789, nP30.771, nP30.924SS, nP30.867SS, nP30.783SS, nP30.771SS, nGST-nP30.1008, nGST-nP30.924, nGST-nP30.873, nGST-nP30.789, nGST-nP30.771, nGST-nP30.924SS, nGST-nP 30.867SS, nGST-nP30.783SS, nGST-nP30.771SS and nDi22.RA. Even more preferred recombinant molecules include SV3:nGST-nP 30.771, SV3:nGST-nP30.771SS and SV3:nDi22.RA.

One aspect of the present invention is the use of an alphavirus expression vector to produce a fusion protein comprising a fusion segment joined to a protein heterologous to that alphavirus (i.e., a protein not of the same species as the alphavirus expression vector). The present inventors are not aware of any reports of the production of fusion proteins in alphavirus-based systems. Moreover, although production of GST-based fusion proteins containing GST fusion segments has been reported in E. coli, the inventors believe they are first to successfully produce a GST-based fusion protein in eukaryotic cells. As such, the present invention includes a recombinant molecule including an alphavirus expression vector operatively linked to a nucleic acid sequence encoding a fusion protein, the fusion protein including a fusion segment joined to a protein heterologous to the alphavirus. As heretofore disclosed, preferred fusion segments include glutathione binding domains, metal binding domains, immunoglobulin binding domains and sugar binding domains, with a glutathione binding domain or a poly-histidine segment being more preferred. Also preferred are proteins that include a signal segment capable of directing secretion of the protein from the cell that produced it.

Another preferred embodiment of the present invention is a recombinant molecule including a nucleic acid sequence operatively linked to an alphavirus expression vector, the nucleic acid sequence encoding a Toxoplasma antigen and/or a Dirofilaria antigen that includes a signal segment capable of secreting the antigen from an animal cell transfected by the recombinant molecule. The antigen can further include a fusion segment as heretofore disclosed.

The present invention includes use of recombinant molecules of the present invention to produce compounds capable of protecting an animal from disease caused by a parasite and/or capable of detecting the presence of such a parasite. Protective compounds of the present invention can be produced by a method that includes (a) culturing an animal cell transfected with a recombinant molecule of the present invention to produce the compound; and (b) recovering the compound.

As heretofore stated, transfection includes any means for introducing a nucleic acid sequence, expression vector, recombinant molecule, or packaging vector, into an animal host cell, including, but not limited to transformation, electroporation, microinjection, lipofection, adsorption, and protoplast fusion. Preferred transfection techniques are lipofection and electroporation. Suitable host cells for producing protective compounds in culture include, but are not limited to, mammalian, insect, avian, reptilian and amphibian cells. Preferred host cells include mammalian, insect, and avian cells. More preferred host cells include Chinese hamster ovary cells, baby hamster kidney cells and chicken embryonic fibroblasts. As used herein, transfection can also include infection of an animal host cell by a recombinant alphavirus containing a recombinant molecule of the present invention. A number of host cells are suitable for infection since alphaviruses have such wide host ranges as heretofore disclosed.

The present invention includes a recombinant cell capable of producing at least one compound capable of protecting an animal from disease caused by a parasite. A recombinant cell of the present invention comprises an animal cell transfected with a recombinant molecule capable of directing expression of the compound, the recombinant molecule including at least one nucleic acid sequence encoding the compound operatively linked to an alphavirus expression vector. Preferred recombinant cells of the present invention are capable of producing compounds capable of protecting animals from infection by preferred parasites as heretofore disclosed. More preferred recombinant cells are capable of producing compounds capable of protecting animals from Toxoplasma and/or Dirofilaria infection, such as cells capable of producing heretofore disclosed Toxoplasma and/or Dirofilaria antigens. Particularly preferred recombinant cells are animal cells transfected with recombinant molecules in which SV1, SV2, SV3, SV4, SV5 or SV6 is operatively linked to at least one of the following nucleic acid sequences: nP30.1008, nP30.924, nP30.873, nP30.789, nP30.771, nP30.924SS, nP30.867SS, nP30.783SS, nP30.771SS, nGST-nP 30.1008, nGST-nP30.924, nGST-nP30.873, nGST-nP30.789, nGST-nP30.771, nGST-nP30.924SS, nGST-nP30.867SS, nGST-nP 30.783SS, nGST-nP30.771SS and nDi22.RA. Even more preferred recombinant cells are animal cells transfected with SV3:nGST-nP30.771, SV3:nGST-nP30.771SS or SV3:nDi22.RA.

In order to produce protective compounds of the present invention, a recombinant cell, produced as described above, is cultured in an effective medium, using techniques such as those described in Xiong et al., ibid. As used herein, an effective medium refers to any medium in which the transfected cells can produce protective compounds of the present invention. An effective medium is typically an aqueous medium comprising assimilable carbohydrate, nitrogen and phosphate sources, as well as appropriate salts, minerals, metals and other nutrients, such as vitamins, growth factors and other hormones. The medium may comprise complex nutrients or may be a defined medium. Recombinant cells of the present invention can be cultured in conventional fermentation bioreactors, which include, but are not limited to, batch, fed-batch, cell recycle and continuous fermentors. Culturing can also be conducted in shake flasks, test tubes, microtiter dishes and petri plates. Culturing is carried out at a temperature, pH and oxygen content appropriate for the recombinant cell. Such culturing conditions are well within the expertise of one of ordinary skill in the art. Examples of preferred effective media and culturing conditions are included in the Examples section.

Depending on whether expression results in a protective protein having or lacking a signal segment, the resultant protein may be secreted into the medium or remain within the recombinant cell. The phrase “recovering the protein” refers simply to collecting the whole fermentation medium (including cells) containing the protein and can, but need not, entail additional steps of separation or purification. Protective compounds of the present invention can be purified using a variety of standard protein or RNA purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

Isolated protective compounds of the present invention are preferably retrieved in “substantially pure” form. As used herein, “substantially pure” refers to a purity that allows for the effective use of the compound as a therapeutic composition or diagnostic. A vaccine for animals, for example, should exhibit no substantial toxicity and should be capable of stimulating the production of antibodies in a vaccinated animal. Preferred isolated compounds of the present invention include protective proteins, with Toxoplasma and Dirofilaria antigens being more preferred. Particularly preferred isolated proteins include P30.336, P30.308, P30.291, P30.289, P30.263, P30.261, P30.257, GST-P30.336, GST-P 30.308, GST-P30.291, GST-P30.289, GST-P30.263, GST-P 30.261, GST-P30.257 and Di22.RA.

Protective compounds made in accordance with the present invention have a variety of uses including, but not limited to, use as vaccines and other therapeutic compounds, use as diagnostic agents and use as antigens in the production of polyclonal or monoclonal antibodies.

One embodiment of the present invention is a therapeutic composition capable of protecting an animal from disease caused by a parasite when the composition is administered to the animal in an effective amount. Therapeutic compositions of the present invention are produced by a method including (a) culturing a recombinant cell of the present invention to produce a compound capable of protecting the animal from the disease, (b) recovering the compound, and (c) formulating a therapeutic composition therefrom. A therapeutic composition of the present invention can include one or more of the following protective compounds: (a) a protective protein of the present invention, (b) a protective RNA species of the present invention and/or (c) an antibody raised against a protective compound of the present invention, as described in more detail below. A multivalent therapeutic composition containing multiple protective compounds targeting multiple parasites can be produced by combining one or more protective compounds after production, by culturing more than one recombinant cell in a culturing reaction or by producing more than one protective compound in a recombinant cell by, for example, transfecting an animal cell with one or more recombinant molecules and/or by transfecting an animal cell with a recombinant molecule containing more than one nucleic acid sequence encoding one or more protective compounds of the present invention. Preferred therapeutic compositions include one or more of the preferred protective compounds heretofore disclosed targeted against one or more of the preferred parasites heretofore disclosed.

Therapeutic compositions of the present invention can be administered to any animal; preferably to mammals, insects and birds; and more preferably to humans, pigs, sheep, dogs, cats, cattle, horses, poultry, ferrets, and other pets and/or economic food animals. Particularly preferred animals to protect include humans, dogs and cats.

Therapeutic compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m or o-cresol, formalin and benzyl alcohol. Standard formulations will either be liquid injectables or solids which can be taken up in a suitable liquid as a suspension or solution for injection. Thus, in a non-liquid formulation, the excipient may comprise dextrose, human serum albumin, preservatives, etc., to which sterile water or saline could be added prior to administration.

In one embodiment of the present invention, the therapeutic composition can also include an immunopotentiator, such as an adjuvant or a carrier. Suitable adjuvants or carriers include the adjuvants and carriers suitable for administration of recombinant virus particle vaccines of the present invention.

In order to protect an animal from disease caused by a parasite, a therapeutic composition of the present invention is administered to the animal in an effective manner such that the composition is capable of protecting that animal from the targeted disease. For example, an isolated protective protein of the present invention, when administered to an animal in an effective manner, is able to elicit (i.e., stimulate) an immune response, preferably including both a humoral and cellular response, that is sufficient to protect the animal from disease. Similarly, an antibody of the present invention, when administered to an animal in an effective manner, is administered in an amount so as to be present in the animal at a titer that is sufficient to protect the animal from disease, at least temporarily. Protective RNA species of the present invention can also be administered in an effective manner, thereby reducing expression of parasitic proteins in order to interfere with parasite development or to produce immunogens capable of eliciting a protective immune response.

Therapeutic compositions of the present invention can be administered to animals-prior to parasite infection in order to prevent infection and/or can be administered to animals after parasite infection in order to treat disease caused by the parasite. For example, protective proteins and antibodies thereof can be used as immunotherapeutic agents.

Acceptable protocols to administer therapeutic compositions in an effective manner include individual dose size, number of doses, frequency of dose administration, and mode of administration. Determination of such protocols can be readily accomplished by those skilled in the art. A suitable single dose is a dose that is capable of protecting an animal from parasitic infection or disease when administered one or more times over a suitable time period. For example, a preferred single dose of a protective protein or antibody therapeutic composition is from about 1 microgram (μg) to about 10 milligrams (mg) of the therapeutic composition for an animal about the size of an average size dog. Booster vaccinations can be administered from about 2 weeks to several years after the original administration. Preferably booster vaccinations are administered when the immune response of the animal becomes insufficient to protect the animal from parasitic infection. A preferred administration schedule is one in which from about 10 μg to about 1 mg of the vaccine per kg body weight of the animal is administered from about one to about two times over a time period of from about 2 weeks to about 12 months. Modes of administration can include, but are not limited to, subcutaneous, intradermal, intravenous, nasal, oral, transdermal and intramuscular routes.

According to one embodiment, a protective RNA species of the present invention can also be administered to an animal in a fashion to enable expression of the nucleic acid sequence into a protective protein in the animal to be protected from parasitic disease. An RNA species can be delivered in a variety of methods including, but not limited to, direct injection (e.g., as “naked” RNA molecules, such as is taught, for example in Wolff et al., 1990, Science 247, 1465-1468), packaged in a viral coat to form a recombinant virus, and packaged as a recombinant cell vaccine. RNA species packaged in a viral coat can, for example, comprise packaging-defective or packaging-competent recombinant molecules. Recombinant virus particle vaccines of the present invention are examples of packaging-defective recombinant molecules packaged in viral coats. An example of a packaging-competent recombinant molecule packaged in a viral coat is presented in the Examples. Therapeutic compositions packaged in a viral coat can be administered using methods as disclosed for the administration of recombinant virus particle vaccines of the present invention.

A recombinant cell vaccine of the present invention includes recombinant cells of the present invention that express at least one protective protein. Such recombinant cells can be administered in a variety of ways known to those skilled in the art, preferably at doses ranging from about 10⁸ to about 10¹² cells per kilogram body weight. Administration protocols are similar to those described herein for protein-based therapeutic compounds. A preferred recombinant cell vaccine comprises a recombinant cell to which at least one protective protein of the present invention is attached due to the protective protein including a hook that prevents the protective protein from being secreted from the cell after production.

The efficacy of a therapeutic composition of the present invention to protect an animal from disease caused by a parasite can be tested in a variety of ways including, but not limited to, detection of protective antibodies (using, for example, protective proteins of the present invention), detection of cellular immunity within the treated animal, or challenge of the treated animal with the targeted parasite or antigens thereof to determine whether the treated animal is resistant to disease. Such techniques are known to those skilled in the art.

One preferred embodiment of the present invention is a method to protect an animal from toxoplasmosis by administering to the animal an effective amount of a therapeutic composition produced by a method including (a) culturing an animal cell transfected with a recombinant molecule to produce a T. gondii P30 antigen, the recombinant molecule comprising at least one nucleic acid sequence encoding the antigen operatively linked to an alphavirus expression vector; (b) recovering the antigen; and (c) formulating a therapeutic composition therefrom that can be administered in an effective amount to protect the animal from toxoplasmosis. Preferably the antigen is a modified T. gondii P30 antigen lacking, amino and/or carboxyl terminal hydrophobic residues, such as P30.336, P30.308, P30.291, P30.289, P30.263, P30.261, P30.257, GST-P 30.336, GST-P30.308, GST-P30.291, GST-P30.289, GST-P 30.263, GST-P30.261, GST-P30.257 or a combination thereof.

Another preferred embodiment of the present invention is a method to protect an animal from heartworm by administering to the animal an effective amount of a therapeutic composition produced by a method including (a) culturing an animal cell transfected with a recombinant molecule to produce a D. immitis antigen, the recombinant molecule comprising at least one nucleic acid sequence encoding the antigen operatively linked to an alphavirus expression vector; (b) recovering the antigen; and (c) formulating a therapeutic composition therefrom that can be administered in an effective amount to protect the animal from heartworm. Preferably the therapeutic compositions contains at least one of the following proteins: D. immitis P39, D . immitis P22L, D . immitis P22U, D . immitis P20.5, D. immitis P4, D. immitis Di22 and/or D. immitis proteases expressed in L3 and/or L4 larvae, or a protein sharing significant homology with such D. immitis proteins. Particularly preferred antigens to protect against heartworm included D. immitis Di22.RA, P39, P22L and/or P20.5.

The present invention also includes antibodies capable of selectively binding to a protective compound of the present invention. Such antibodies can be either polyclonal or monoclonal antibodies. Antibodies of the present invention include functional equivalents such as antibody fragments and genetically-engineered antibodies, including single chain antibodies, that are capable of selectively binding to at least one of the epitopes of a protective compound of the present invention.

A preferred method to produce antibodies of the present invention includes (a) administering to an animal an effective amount of a protective compound of the present invention to elicit an immune response and (b) recovering the antibodies. Antibodies raised against defined compounds can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. For example, such antibodies can be used (a) as therapeutic compositions to passively immunize an animal in order to protect the animal from parasite disease, (b) as reagents in assays to detect parasite infection, and/or (c) as tools to recover desired parasite proteins from a mixture of proteins and other contaminants. Furthermore, antibodies of the present invention can be used to target cytotoxic agents to parasite in order to directly kill parasites expressing proteins selectively bound by the antibodies. Targeting can be accomplished by conjugating (i.e., stably joining) such antibodies to the cytotoxic agents.

It is also within the scope of the present invention to use protective compounds of the present invention and antibodies thereof as diagnostic agents. One embodiment is a diagnostic assay capable of detecting infection by a parasite, the assay including a parasitic antigen produced by a method including (a) culturing a recombinant cell containing a recombinant molecule of the present invention that encodes the antigen to produce the antigen, and (b) recovering the antigen. The assay also includes a means for detecting the binding of an antibody indicative of parasite infection to the parasite antigen. Preferably the assay contains Toxoplasma and/or Dirofilaria antigens capable of detecting toxoplasmosis or heartworm, respectively. Another embodiment is a diagnostic assay that detects parasite antigens using antibodies as disclosed in the present invention. The present invention also includes methods to detect parasite diseases, such as toxoplasmosis, in animals using such assays. For example, one can contact a bodily fluid of the animal with a T. gondii P30 antigen produced in accordance with the present invention (i.e., using an alphavirus expression system) and detect toxoplasmosis by determining the ability of the antigen to form a selective complex with antibodies in the bodily fluid. Preferably the antigen is a modified T. gondii P30 antigen lacking-amino and/or carboxyl terminal hydrophobic residues, such as P30.336, P30.308, P30.291, P30.289, P30.263, P30.261, P30.257, GST-P30.336, GST-P 30.308, GST-P30.291, GST-P30.289, GST-P30.263, GST-P 30.261, GST-P30.257 or a combination thereof.

The following experimental results are provided for purposes of illustration and are not intended to limit the scope of the invention.

### EXAMPLES

### Example 1

This example describes the production of nucleic acid sequences encoding recombinant T. gondii P30 antigens.

A. An approximately 1020 base pair DNA fragment, called nP30.1008, shown in FIG. 3, containing the entire coding region for the T. gondii P30 antigen (about 336 amino acids, assuming that the translation initiation site is as shown in Table 1) and flanked by SmaI and XbaI restriction endonuclease sites immediately adjacent to the translation initiation (start) site and XhoI, KpnI, and MluI restriction endonuclease sites immediately adjacent to the translation termination (stop) site was copied from a clone (Burg et al., ibid.) containing the P30 gene with natural flanking sequence using polymerase chain reaction (PCR) amplification (Sambrook et al., ibid.). The specific primers used in the amplification reaction were:

```
Primer #1
5′ CCCGGGTCTA GA ATG TCG GTT TCG CTG CAC CAC 3′
      SmaI XbaI 1st ATG at amino terminus of P30
Primer #2
5′ ACGCGTGGTA CCTCGAG TCA CGC GAC ACA AGC T 3′
      MluI KpnI XhoI Translation Stop Site
```

The nucleic acid sequence nP30.1008 was recovered, restricted with XbaI and XhoI, and inserted into plasmid pBluescript II (a cloning vector available from Stratagene, San Diego, Calif.) which had also been restricted with XbaI and XhoI. The resulting vector, referred to as pB:nP30.1008, encodes T. gondii antigen P30.336.

B. A nucleic acid sequence encoding a functionally equivalent P30 antigen, lacking both amino and carboxyl terminal hydrophobic residues, was produced by the following method. A fragment of about 800 base pairs was copied by PCR amplification of a portion of bP:nP30.1008 (produced as described in Example 1A) using the following primers:

```
Primer #3
5′ GTCGACCCCG GGTCTAGACC ATG GCA TCG GAT CCC CC 3′
      SalI SmaI XbaI NcoI
                                                  Translation Start Site
Primer #4
5′ ACGCGTGGTA CCTCGAG TTA TGC TGA CCC TGC AGC CCC
```

GGC

KpnI XhoI Translation Stop Site

The amplified nucleic acid sequence, denoted nP30.789, is shown in FIG. 3. nP30.789 encodes P30.263, a protective protein, the primary translation product of which is about 263 amino acids, spanning amino acid from about 46 through amino acid about 308, as numbered in the deduced amino acid sequence of Table 1. As such, about 45 amino acid residues have been removed from the amino terminus and about 28 amino acid residues have been removed from the carboxyl terminus of P30.336.

C. A nucleic acid sequence encoding a functionally equivalent P30 antigen, lacking amino terminal hydrophobic residues, is produced by the following method. A fragment of about 880 base pairs is copied by PCR amplification of a portion of bP:nP30.1008 (produced as described in Example 1A) using primer #3 (described in Example 1B) and primer #2 (described in Example 1A). The amplified nucleic acid sequence, called nP30.873, is shown in FIG. 3. nP30.873 encodes P30.291, a protective protein, the primary translation production of which is about 291 amino acids, spanning amino acid from about 46 through amino acid about 336, as numbered in the deduced amino acid sequence of Table 1. As such, about 45 amino acid residues are removed from the amino terminus of P30.336.

D. Another nucleic acid sequence encoding a functionally equivalent P30 antigen, lacking amino terminal hydrophobic residues, was produced by the following method. A fragment of about 880 base pairs was copied by PCR amplification of a portion of bP:nP30.1008 (produced as described in Example 1A) using the following primers:

```
Primer #5
5′ GTCGACTCTA GACCCGGG ATG GCA TCG GAT CCC CC 3′
                                  SmaI Translation Start Site
Primer #6
5′ ACGCGTCGTA CCGAATTCTCA CGC GAC ACA AGC T 3′
                                  EcoRI Translation Stop Site
```

This amplified nP30.873 nucleic acid sequence is similar to the nP30.873 nucleic acid sequence shown in FIG. 3 except that this nucleic acid sequence is flanked by SmaI and EcoRI restriction sites rather than by XbaI and XhoI restriction sites.

E. A nucleic acid sequence encoding a functionally equivalent P30 antigen, lacking carboxyl terminal hydrophobic residues, is produced by the following method. A fragment of about 940 base pairs is copied by PCR amplification from bP:nP30.1008 (produced as described in Example 1A) using primer #1 (described in Example 1A) and primer #4 (described in Example 1B). The amplified nucleic acid sequence, called nP30.924, is shown in FIG. 3. nP30.924 encodes P30.308, a protective protein, the primary translation product of which is about 308 amino acids, spanning amino acid from about 1 through amino acid about 308, as numbered in the deduced amino acid sequence of Table 1. As such, about 28 amino acid residues are removed from the carboxyl terminus of P30.336.

F. A nucleic acid sequence encoding a functionally equivalent P30 antigen, lacking both amino and carboxyl terminal hydrophobic residues, but having a signal sequence in its primary translation product is produced in the following manner. A fragment of about 783 base pairs is produced by PCR amplification of a portion of bP:nP30.1008 (produced as described in Example 1A) using primer #7 and primer #4 (described in Example 1B). Primer #7, shown below, contains a nucleic acid signal segment encoding the first about 23 residues of human t-PA joined to a nucleotide sequence encoding amino acid residues about 48 through about 54 of P30.336.

```
Primer #7
5′ GTCGACCCCG GGTCTAGACC ATG GAT GCA ATG AAG AGA
                                XbaI Translation Start of t-PA
      GGG CTC TGC TGT GTG CTG CTA CTG TGT GGA GCA GTC
      TTC GTT TCG CCC AGC TCG GAT CCC CCT CTT GTT GCC 3′
                              P30 Sequences
```

The amplified nucleic acid sequence, called nP30.783SS, encodes P30.261SS, a protective protein, the primary translation product of which is about 283 amino acids. P30.261SS is capable of being secreted as a protein, which upon secretion from the cell is very similar to P30.263 except that P30.261SS begins at amino acid 48 of P30 as numbered in the deduced amino acid sequence of Table 1, whereas P30.263 begins at amino acid 46. Amino acid 48 is thought to be the amino-terminal amino acid of the mature P30 protein (Burg et al., ibid.).

G. A nucleic acid sequence which encodes a fusion segment (i.e., glutathione S-transferase, or GST) joined to a T. gondii antigen lacking a majority of amino and carboxyl terminal hydrophobic sequences was produced in the following manner.

P30.5 COS1.1 DNA, a clone containing the entire T. gondii antigen gene (Burg et al., ibid.) was digested with BamHI and PstI in order to obtain a BamHI/PstI fragment of about 771 base pairs which encodes P30 antigen containing amino acid residues spanning from about 49 through about 305, as numbered in the deduced amino acid sequence of Table 1. The BamHI/PstI fragment was inserted into plasmid pBluescript II (available from Stratagene, San Diego, Calif.) which had also been digested with BamHI and PstI. The resulting vector, referred to as pB:nP30.771, encodes T. gondii antigen P30.257. Vector pB:nP30.771 was digested with BamHI and EcoRI to obtain a BamHI/EcoRI fragment of about 773 base pairs that was subsequently inserted into the GST fusion vector pGEX-1 (Smith et al., pp. 31-40, 1988, Gene, Vol. 67) which had also been digested with BamHI and EcoRI. The resultant vector, denoted pGEX:nP30.771, contained the nP30.771 coding region immediately adjacent to and in the same reading frame as the GST gene, the transcription of which was under the control of the tac promoter.

A nucleic acid sequence of about 703 base pairs containing the GST coding region joined to the nP30.771 coding region, called nGST-nP30.771, was produced by PCR amplification of a portion of the pGEX:nP30.771 vector using the primers shown below:

Primer #8
5′ GTCGACCCCG GG TCTAGACC ATG TCC CCT ATA CTA GG

3′

```
                                      XbaI Translation Start Site
Primer #9
5′ ACGCGTCGTA CCTCGAG TCA G TCA G TCA CGATG 3′
                                  XhoI Translation Stop Sites
```

As indicated, primer #8 spans the GST translation start site of pGEX-1, and primer #9 spans the three translation stop sites in pGEX-1. The amplified nucleic acid sequence nGST-nP30.771 encodes a GST fusion protein in which GST is joined to a P30 protein of about 257 amino acids, the P30 protein lacking the initial about 48 amino acids and the terminal about 31 amino acids of P30.336.

Additional nucleic acid sequences encoding GST-based fusion proteins either lacking a signal segment (e.g., nGST-nP30.1008, nGST-nP30.924, nGST-nP30.873 and nGST-nP 30.789 and/or containing a t-PA signal segment (e.g., nGST-nP30.924SS, nGST-nP30.867SS, nGST-nP30.783SS and nGST-nP 30.771SS can be produced using similar techniques.

### Example 2

This example describes the production of certain recombinant molecules containing nucleic acid sequences encoding P30 antigens.

A. A recombinant molecule containing a nucleic acid sequence encoding a full-length T. gondii P30 antigen is produced by the following method. Sindbis virus expression vector TRCAT62 (see Xiong et al., ibid.) is a double-stranded DNA expression vector which contains (a) the genes encoding nonstructural Sindbis virus polypeptides nsP1, nsP2, nsP3, and nsP4, (b) the subgenomic promoter plus 14 nucleotides downstream from the subgenomic RNA initiation site which is ligated to a gene encoding chloramphenicol acetyltransferase (CAT), (c) 62 nucleotides at the 3′ end of the Sindbis viral genome plus the poly(A) tail, and (d) a bacteriophage SP6 promoter immediately upstream of the Sindbis virus nonstructural genes. The gene encoding CAT is flanked by an XbaI restriction site at its 5′ end and an XhoI restriction site at its 3′ end.

Double stranded DNA recombinant molecule dSV2:nP30.1008 is produced in the following manner. TRCAT62 is digested with the restriction enzymes XbaI and XhoI, thereby removing the CAT gene and forming dSV2 (see FIG. 4). Nucleic acid sequence nP30.1008 (produced as described in Example 1A and shown in FIG. 3) is digested with XbaI and XhoI and subsequently ligated into dSV2 to form DNA recombinant molecule dSV2:nP30.1008.

RNA recombinant molecule SV2:nP30.1008 is produced from dSV2:nP30.1008 by digesting dSV2:nP30.1008 DNA with restriction enzyme MluI and incubating the digested DNA with bacteriophage SP6 RNA polymerase under conditions similar to those described by Xiong et al., ibid., and Rice et al., J. Virology 61, 3809-3819, 1987, in order to produce run-off transcripts comprising SV2:nP30.1008. Trace quantities of ³H-UTP (uridine triphosphate) or α-³²p-CTP (cytosine triphosphate) are included in the transcription reaction to permit quantitation (i.e., using DE81 filter paper, available from Whatman Inc., Clifton, N.J.) and gel analysis of the RNA transcripts.

B. Recombinant molecule SV3:nGST-nP30.771 was produced as follows. An expression vector containing nGST-nP 30.771 was produced by (a) digesting nGST-nP30.771 (produced as described in Example 1G) with XbaI and XhoI; b) inserting the XbaI/XhoI fragment into expression vector Toto2J1 which had also been digested with XbaI and XhoI, to form DNA expression vector dSV3:nGST-nP30.771; (c) digesting dSV3:nGST-nP30.771 with MluI to form a linear molecule; and (d) transcribing the linear molecule using bacteriophage SP6 RNA polymerase as described in Example 2A to obtain RNA expression vector SV3:nGST-nP30.771. Note that Toto2J1 is a Sindbis virus expression vector that contains the SP6 RNA polymerase promoter and the entire Sindbis virus genome through to the NsiI restriction site at nucleotide 11452 (i.e., each of the nonstructural polypeptide genes, the subgenomic promoter, and each of the structural polypeptide genes) ligated to an SspI (nucleotide position 7499)/SstI restriction fragment from TRCAT62 which contains the subgenomic promoter, 14 nucleotides of the 5′ untranslated sequence of the subgenomic mRNA, the CAT gene, 62 nucleotides of Sindbis virus 3′ untranslated sequence, and the Sindbis virus poly-A sequence.

C. A number of other RNA recombinant molecules can be produced as described above, including other recombinant molecules comprising SV1, SV2, SV3, SV4, SV5 or SV6 operatively linked to at least one of the following nucleic acid sequences: nP30.1008, nP30.924, nP30.873, nP30.789, nP30.771, nP30.924SS, nP30.867SS, nP30.783SS, ,nP30.771SS, nGST-nP30.1008, nGST-nP30.924, nGST-nP30.873, nGST-nP 30.789, nGST-nP30.771, nGST-nP30.924SS, nGST-nP30.867SS, nGST-nP30.783SS and nGST-nP30.771SS.

### Example 3

This Example describes a method to produce a Sindbis virus packaging vector of the present invention. The example provided herein comprises a packaging vector containing the genetic information to encode Sindbis virus structural polypeptides and control signals for replication and transcription but not containing the site located within the nsP1 gene thought to be required for packaging. Therefore, the packaging vector cannot effect self-packaging.

A full-length cDNA copy of Sindbis virus vector Toto1000 (Rice et al., ibid.) is subjected to partial digestion by the restriction endonuclease SspI, which has cleavage sites at about nucleotides 504, 4130, 7499, and 11954. The desired SspI restriction fragment, which includes a span of nucleotides from about nucleotide 7499 through about nucleotide 504, including nucleotide “0” as indicated in FIG. 5, is isolated, using low melt agarose gel chromatography and elution techniques as described in Sambrook et al., ibid. The desired fragment is then self-ligated using standard ligation technology. The resultant vector, which represents the DNA copy of the packaging vector and is referred to as dPV1, contains the subgenomic promoter, all of the Sindbis virus structural genes, replication and transcription signals at the 5′ and 3′ ends of the linear viral RNA genome, and a bacteriophage SP6 promoter but does not contain the site thought to be required for packaging in the nsP1 gene or genes that encode functional nsP1, nsP2, nsP3, or nsP4 polypeptides. Thus, the packaging vector can work as a “helper” to provide structural polypeptides “in trans” to enable packaging of recombinant molecules of the present invention.

RNA packaging vector PV1 is produced in a manner similar to the RNA recombinant molecules described in Example 2. Briefly, dPV1 is digested with restriction enzyme SstI, and the linearized DNA is incubated with bacteriophage SP6 RNA polymerase. The resultant RNA is referred to PV1.

### Example 4

This Example describes the production of a recombinant virus particle vaccine of the present invention.

A recombinant virus particle vaccine is produced by co-transfecting a host cell with a recombinant molecule and an alphavirus packaging vector, culturing the host cell in an effective medium to produce the vaccine, and recovering the vaccine.

In one experiment, recombinant virus particle vaccine VPV SV2:nP30.1008 is produced by co-transfecting baby hamster kidney (BHK) cells with recombinant molecule SV2:nP30.1008 and packaging vector PV1 using electroporation in a manner similar to that described by Liljestrom et al., ibid. Briefly, BHK cells are grown in 60 mm tissue culture plates to a monolayer confluency of about 90%. The cells are trypsinized, washed once with Minimal Essential Medium (also called MEM; available from Life Technologies Inc., Gaithersburg, Md.) containing 10% fetal calf serum, washed once with ice cold phosphate buffered saline (8 g NaCl, 0.2 g KCl, 1.44 g Na₂HPO₄, 0.24 g KH₂PO₄ per liter of water, the pH of which is adjusted to about pH 7.4; also called PBS) and resuspended in PBS at about 1×10⁷ cells per ml. About 0.5 ml of cells and about 5-10 μg (in about 10-50 microliters (μl)) total of SV2:nP30.1008 and PV1 (at a mole/mole ratio of about 1:1) are mixed in a 0.2 centimeter (cm) cuvette suitable for use in Bio-Rad's Gene Pulser Apparatus (both available from Bio-Rad Laboratories, Richmond, Calif.). The RNA either may be used directly from the in vitro transcription reaction mixture (as described in Example 2 for the recombinant molecule and in Example 3 for the packaging vector) or may be diluted with transcription buffer containing 5 millimolar (mM) dithiothreitol and 1 unit of RNasin per ml. Electroporation is conducted at room temperature by two consecutive pulses at 1.5 kilovolts (KV) and 35 microfarads (μF), using the Gene Pulser Apparatus with the pulse controller unit set at maximum resistance. After electroporation, the cells are diluted about 1:20 in complete BHK cell medium and transferred to tissue culture plates. The cells are then cultured for about 24 to about 36 hours at about 37° C. and about 5% carbon dioxide in about 5 ml of MEM with 10% fetal calf serum.

Plaque forming units (pfu) are quantified by overlaying the monolayers of BHK cells with 2 ml of 1.2% Seakem agarose (available from FMC Corp., Marine Colloids Div., Rockland, Me.) diluted 1:1 (vol/vol) in MEM and 2% fetal calf serum, incubating at about 37° C. for about 24 to about 48 hours, and staining with neutral red or crystal violet.

VPV SV2:nP30.1008 is recovered from the culture using Matrex® Cellufine™ Sulfate Media & Virus Recovery System, available from Amicon Inc., Danvers, Mass.

### Example 5

This Example describes the effect of administering a recombinant virus particle vaccine of the present invention to mice.

Recombinant virus particle vaccine VPV SV2:nP30.1008, produced as described in Example 4, is injected into CD-1 mice using the following protocol. The vaccine is mixed with Hanks' Balanced Salt Solution (HBSS; available from Life Technologies Inc., Gaithersburg, Md.) to give a vaccine formulation of about 1×10⁵ pfu of VPV SV2:nP30.1008 per ml formulation. Each mouse is injected subcutaneously with approximately 0.1 ml of the vaccine formulation at day 0 and at about days 21 to 28. Control CD-1 mice are administered an equivalent amount of native Sindbis virus in HBSS.

The ability of the mice to produce antibodies against T. gondii parasites is measured using an enzyme-linked immunoassay (ELISA). Purified sonicated parasites are placed in microtiter plates and blocked with 5% Fetal Bovine Serum (FBS). Sera collected from mice are incubated for 2 hr at 37° C. in the parasite-coated microtiter wells and washed with PBS containing 0.4 % of the nonionic detergent Tween 2Q. Anti-T. gondii antibodies present in the serum are identified using peroxidase-labeled goat anti-mouse IgG antibodies (available from Cappell Laboratories, Cochranville, Pa.) in a standard ELISA.

The ability of VPV SV2:nP30.1008 to protect the mice from T. gondii infection is determined as follows. Immunized mice are challenged intraperitoneally with about 5×10⁵ T. gondii C strain tachyzoites per mouse. Mice are monitored twice a day until signs of lethal toxoplasmosis are evident at which time the mice are euthanized with an overdose of metaphane. After 30 days post challenge, all surviving mice are euthanized with an overdose of metaphane and the number of brain cysts determined by removing the brains from the animals, gently homogenizing the brain tissue in PBS, and counting cysts in 10μl samples in a hemacytometer. CD-1 mice vaccinated with VPV SV2:nP30.1008 show few if any brain cysts upon infection by T. gondii, especially as compared to mice vaccinated with Sindbis virus.

Thus, VPV SV2:nP30.1008 is capable of protecting mice from T. gondii infection. Mice are a suitable model for T. gondii infection studies since the chronology and outcome of infection in most warm-blooded animals is very similar.

### Example 6

This Example describes the production of a recombinant cell capable of expressing a modified P30 antigen and use of the recombinant cell to produce an antigen capable of eliciting an immune response against T. gondii and antigens thereof.

The ability of GST-P30.257, the fusion protein encoded by nucleic acid sequence nGST-nP30.771, to elicit an immune response against toxoplasma infection was determined as follows.

A recombinant cell capable of expressing the GST-P 30.257 fusion protein was produced by transfecting SV3:nGST-nP30.771 into baby hamster kidney (BHK) cells using electroporation in a manner similar to that described by Liljestrom et al., ibid. Briefly, BHK cells were grown in 60 mm tissue culture plates to a monolayer confluency of about 90%. The cells were trypsinized, washed once with Minimal Essential Medium (also called MEM; available from Life Technologies Inc., Gaithersburg, Md.) containing 10% fetal calf serum, washed once with ice cold phosphate buffered saline (8 g NaCl, 0.2 g KCl, 1.44 g Na₂HPO₄, 0.24 g KH₂P0₄ per liter of water, the pH of which is adjusted to about pH 7.4; also called PBS) and resuspended in PBS at about 1×10⁷ cells per ml. About 0.5 ml of cells and about 5-10 μg (in about 10-50 μl) of SV3:nGST-nP30.771 were mixed in a 0.2 centimeter (cm) cuvette suitable for use in Bio-Rad's Gene Pulser Apparatus (both available from Bio-Rad Laboratories, Richmond, Calif.). The RNA was either used directly from the in vitro transcription reaction mixture (as described in Example 2) or was diluted with transcription buffer containing 5 millimolar (MM) dithiothreitol and 1 unit of RNasin per ml. Electroporation was conducted at room temperature by two consecutive pulses at 1.5 kilovolts (KV) and 35 microfarads (μF), using the Gene Pulser Apparatus with the pulse controller unit set at maximum resistance. After electroporation, the cells were diluted about 1:20 in complete BHK cell medium and transferred to tissue culture plates.

Transfected cells were then cultured in MEM medium with 10% fetal calf serum for about 24 to about 36 hours at about 37° C. in order to produce GST-P30 recombinant virus. BHK cells were infected by GST-P30 recombinant virus and incubated for about 12 to about 16 hours at 37° C. to produce GST-P30.257. The GST-P30.257 that was expressed from SV3:nGST-nP30.771 was specifically recognized by (i.e., selectively bound with high affinity to) polyvalent antiserum produced against native T. gondii P30 protein, polyvalent antiserum produced against T. gondii tachyzoite cell lysate, and P30 monoclonal antibodies 1G5 and 5D12 in immunoblot analysis experiments. Rabbit polyvalent antisera produced against native P30 protein or against tachyzoite cell lysates were obtained from L. H. Kasper, Department of Medicine and Microbiology, Section of Neurology, Dartmouth Medical School, Hanover, N.H. P30 monoclonal antibodies 1G5 and 5D12 were obtained from J. S. Remington, Department of Immunology and Infectious Diseases, Research Institute, Palo Alto Medical Foundation, Palo Alto, Calif. ELISA protocols are discussed in Example 5.

GST-P30.257 was purified by glutathione sepharose 4B chromatography (resin available from Pharmacia Biotech Inc., Piscataway, N.J.) using a technique similar to that described by Smith et al., ibid. The purified protein was used to immunize a rabbit according to the following protocol. About 40 μg of GST-P30.257 was injected into a rabbit on days 0, 21, and 42. Serum was collected from the rabbit at day 56 and found to react with (i.e., bind to) both native T. gondii P30 antigen and T. gondii tachyzoite parasites in an immunoblot assay, indicating that GST-P 30.257 was capable of eliciting an immune response against toxoplasma infection.

### Example 7

This Example demonstrates the ability of a modified T. gondii P30 antigen to protect an animal from toxoplasmosis.

Protective protein CST-P30.257, produced as described in Example 6, is injected into CD-1 mice using the following protocol. CST-P30.257 is formulated with an adjuvant (e.g., Freund's complete adjuvant, Freund's incomplete adjuvant, liposomes or Quil A), using standard formulation protocols, to produce a therapeutic composition. Each mouse is injected subcutaneously with approximately 0.1 ml of the therapeutic composition (including about 10 μg of GST-P30.257) at day 0 and at about day 14. Negative control CD-1 mice are administered adjuvant alone and/or with GST, whereas positive control mice are administered native T. gondii P30 antigen formulated with liposomes, which has been shown by Bulow et al., ibid., to protect mice from Toxoplasma infection.

The ability of the mice to produce antibodies against T. gondii parasites is measured using an enzyme-linked immunoassay (ELISA). Purified sonicated parasites are placed in microtiter plates and blocked with 5% Fetal Bovine Serum (FBS) . Sera collected from mice are incubated for 2 hr at 37° C. in the parasite-coated microtiter wells and washed with PBS containing 0.4 % of the nonionic detergent Tween 20. Anti-T. gondii antibodies present in the serum are identified using peroxidase-labeled goat anti-mouse IgG antibodies (available from Cappell Laboratories, Cochranville, Pa.) in a standard ELISA.

The ability of GST-P30.257 to protect the mice from T. gondii infection is determined as follows. GST-P30.257 immunized mice are challenged intraperitoneally with about 5×10⁵ T. gondil C strain tachyzoites per mouse. Mice are monitored twice a day until signs of lethal toxoplasmosis are evident at which time the mice are euthanized with an overdose of metaphane. After 30 days post challenge, all surviving mice are euthanized with an overdose of metaphane and the number of brain cysts determined by removing the brains from the animals, gently homogenizing the brain tissue in PBS, and counting cysts in 10μl samples in a hemacytometer. CD-l mice vaccinated with GST-P30.257 plus adjuvant show few if any brain cysts upon infection by T. gondii, especially as compared to mice vaccinated with adjuvant alone.

Thus, GST-P30.257 is capable of protecting mice from T. gondii infection. Mice are a suitable model for T. gondii infection studies since the chronology and outcome of T. gondii infection in most warm-blooded animals is similar.

### Example 8

This Example describes the production of nucleic acid sequences encoding recombinant D. immitis Di22 antigens.

A nucleic acid sequence encoding a D. immitis Di22 antigen (included in GeneBank data base accession number M82811) contains a nucleotide repeat of 399 base pairs that encodes a peptide of 133 amino acids, referred to as Di22.RA. A nucleic acid sequence encoding Di22.RA flanked by an XbaI restriction endonuclease site adjacent to the translation initiation (start) site and an XhoI restriction endonuclease site adjacent to the translation termination (stop) site was copied from the Di22 nucleic acid sequence using PCR amplification. The specific primers used in the amplification reaction were:

Primer #10
5′ GTCGACCCCG GGTCTAGAAC C ATG GCT CTC AGT GAA ATC
XbaI 1st ATG at amino ter-

A 3′
minus of RA

Primer #11
5′ ACGCGTGGTA CCTCGAG TCA TCT GCA GCC TTC TTG AA
XhoI Translation Stop Site

3′

The resultant nucleic acid sequence, referred to as nDi22.RA, was recovered, restricted with XbaI and XhoI, and inserted into plasmid pBluescript II, which had also been restricted with XbaI and XhoI. The resulting vector, referred to as pB:nDi22.RA, encodes the D. immitis antigen Di22.RA.

### Example 9

This Example describes the production of a recombinant molecule containing a nucleic acid sequence encoding a D. immitis Di22 antigen.

Recombinant molecule SV3:nDi22.RA was produced by (a) digesting pB:nDi22.RA (described in Example 8) with XbaI and XhoI; (b) inserting the nDi22.RA XbaI/XhoI fragment into expression vector Toto2J1 (described in Example 2B) which had also been digested with XbaI and XhoI, to form DNA expression vector dSV3:nDi22.RA; (c) digesting dSV3:nDi22.RA with MluI to form a linear molecule; and (d) transcribing the linear molecule using bacteriophage SP6 RNA polymerase as described in Example 2A to obtain RNA expression vector SV3:nDi22.RA.

### Example 10

This Example describes the production of a recombinant cell capable of expressing a D. immitis Di22 antigen and use of the recombinant cell to produce an antigen capable of selectively binding to antisera raised against a D. immitis Di22 antigen expressed in E. coli as well as to dog immune serum capable of inhibiting heartworm development; preparation of such dog immune serum is disclosed in Grieve et al., ibid.

A recombinant cell capable of expressing Di22.RA was produced by transfecting SV3:nDi22.RA into BHK cells using lipofection in a manner similar to that described by Feigner et al., pp. 7413-7417, 1987, Pxoc. Natl. Acad. Sci. USA, Vol. 84. Briefly, BHK cells were grown in 60 mm tissue culture plates to a monolayer confluency of about 90%. The cells were washed with PBS and incubated for about 10 minutes at about room temperature with a mixture of from about 0.25 to about 1.0 μg of SV3:nDi22.RA and about 20 μg of Lipofectin (available from Life Technologies Inc., Gaithersburg, Md.) in about 0.4 ml PBS. The mixtures was then removed and the cells washed two times with PBS and incubated with about 5 ml of MEM containing 10% fetal calf serum for about 24 to about 36 hours at about 37° C. in order to produce recombinant virus. BHK cells were infected by the recombinant virus and incubated for about 12 to about 16 hours at about 37° C. to produce Di22.RA.

Recombinant cells were lysed, and the resultant lysates were fractionated by SDS-PAGE, transferred to nitrocellulose paper and incubated either with the immune dog serum referred to above or with rabbit antiserum raised to a Di22 antigen produced by E. coli using standard techniques. Immunoblot analysis of experiments using either serum indicated the presence of a protein of about 15 kD (corresponding to the about 133 amino acid Di22.RA polypeptide) in lysates from cells expressing Di22.RA but not in lysates from cells infected with wild-type Sindbis virus or in lysates from non-infected cells. These results indicate that SV3:nDi22.RA can direct the production of a D. immitis antigen recognized by serum capable of inhibiting heartworm development as well as by antibodies raised against recombinant Di22 produced by bacteria.

### Example 11

This Example describes the production of a therapeutic composition comprising a packaging-competent recombinant molecule capable of expressing a heartworm antigen packaged in a viral coat. This Example also demonstrates the ability of such a therapeutic composition to slow the growth of heartworm larvae.

Packaging-competent recombinant molecule SV3:nDi22.RA, produced as described in Example 9, was packaged into a viral coat as follows. SV3:nDi22.RA was transfected into BHK cells using lipofection in a manner similar to that described by Felgner et al., pp. 7413-7417, 1987, Proc. Natl. Acad. Sci. U.S.A., Vol. 84. Briefly, BHK cells were grown in 60 mm tissue culture plates to a monolayer confluency of about 90%. The cells were washed with PBS and incubated for about 10 minutes at about room temperature with a mixture of from about 0.25 to about 1.0 μg of SV3:nDi22.RA and about 20 μg of Lipofectin (available from Life Technologies Inc., Gaithersburg, Md.) in about 0.4 ml PBS. The mixtures was then removed and the cells washed two times with PBS and incubated with about 5 ml of MEM containing 10% fetal calf serum for about 24 to about 36 hours at about 37° C. in order to produce recombinant virus RV SV3:nDi22.RA, also known as HJA.

Female C57 BL/6J mice, about 6 to 8 weeks old, were inoculated subcutaneously in the inguinal region as follows:

(a) 8 mice were each inoculated with about 5×10⁶ pfu of RV SV3:nDi22.RA in about 0.1 ml of Hanks' Balanced Salt Solution with 1 percent fetal calf serum (HBSS/1% FCS);

(b) 8 mice were each inoculated with about 5×10⁶ pfu of non-recombinant Sindbis virus in about 0.1 ml HBSS/1% FCS; and

(c) 6 mice were each inoculated with 0.1 ml HBSS/1% FCS.

About 14 days after. the initial injection, each mice was boosted with the same dose of the same material as was initially injected. About 35 days after the initial injection, each mouse was implanted with a diffusion chamber containing about 40 D. immitis L3 larvae, according to the technique described by Abraham et al., 1988, J. Parasitol. 74, 275-282. (One mouse administered the recombinant virus died.) The chambers were removed about 56 days after the initial injection and analyzed for larval survival and growth as described in Abraham et al., ibid.

About 65% of the larvae were recovered from chambers implanted on mice administered the recombinant virus RV SV3:nDi22.RA in HBSS/1% FCS (RV larvae), of which about 38% had died. About 61% of the larvae were recovered from chambers implanted on mice administered the non-recombinant Sindbis virus in HBSS/1% FCS (SV larvae), of which about 22% had died. About 72% of the larvae were recovered from chambers implanted on mice administered HBSS/1% FCS along (HBSS larvae), of which about 16% had died. Living RV larvae exhibited significantly stunted growth compared to living SV larvae and living HBSS larvae. That is, living RV larvae exhibited an average length of 370.653±29.249 μm (micrometers) compared to living SV larvae which exhibited an average length of 387.769±39.793 μm (p =0.0045), and to living HBSS larvae which exhibited an average length of 382.321±41.837 μm (p =0.0396). These results indicate that administration of RV SV3:nDi22.RA can lead to the production of a D. immitis antigen capable of stunting larval heartworm growth.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims:

What is claimed is:

1. A recombinant virus particle vaccine comprising a packaging-defective recombinant molecule packaged in an alphavirus coat, said packaging-defective recombinant molecule comprising a nucleic acid sequence that encodes at least one protective compound selected from the group consisting of protective proteins and protective RNA species, said nucleic acid sequence being operatively linked to a packaging-defective alphavirus expression vector that is capable of directing expression of said packaging-defective recombinant molecule.

2. The vaccine of claim 1, wherein said packaging-defective alphavirus expression vector is capable of directing replication of said packaging-defective recombinant molecule.

3. The vaccine of claim 1, wherein an animal having said vaccine administered thereto produces said protective compound.

4. The vaccine of claim 1, wherein said disease is caused by an infectious agent selected from the group consisting of protozoan parasites, helminth parasites, ectoparasites, fungi, bacteria, and viruses.

5. The vaccine of claim 1, wherein said disease is caused by an infectious agent selected from the group consisting of the genera Toxoplasma, Dirofilaria, Acanthocheilonema, Babesia, Brugia, Candida, Cryptococcus, Cryptosporidium, Dipetalonema, Eimeria, Encephalitozoon, Hepatozoon, Histoplasma, Isospora, Loa, Microsporidia, Neospora, Nosema, Onchocerca, Parafilaria, Plasmodium, Pneumocystis, Rochalimaea, Setaria, Stephanofilaria, Theileria and Wuchereria.

6. The vaccine of claim 1, wherein said animal is selected from the group consisting of mammals, insects, and birds.

7. The vaccine of claim 1, wherein said protective compound is capable of eliciting an immune response to protect said animal from said disease.

8. The vaccine of claim 1, wherein said protective compound comprises a Toxoplasma gondii antigen capable of eliciting an immune response to protect said animal from toxoplasmosis.

9. The vaccine of claim 1, wherein said protective compound comprises a Toxoplasma gondii P30 antigen capable of eliciting an immune response to protect said animal from toxoplasmosis.

10. The vaccine of claim 1, wherein said protective compound comprises a Toxoplasma gondii P30 antigen selected from the group consisting of P30.336, P30.308, P30.291, P30.289, P30.263, P30.261, and P30.257.

11. The vaccine of claim 1, wherein said nucleic acid sequence encodes a protective protein further comprising a signal segment capable of promoting secretion of said protective protein.

12. The vaccine of claim 1, wherein said packaging-defective alphavirus expression vector is selected from the group consisting of packaging-defective Sindbis virus expression vectors, packaging-defective Semliki Forest virus expression vectors, packaging-defective Ross River virus expression vectors, packaging-defective Venezuela equine encephalitis virus expression vectors, or wherein said packaging-defective alphavirus expression vector comprises a hybrid of any one or more of said packaging-defective alphavirus expression vectors.

13. The vaccine of claim 1, wherein said packaging-defective alphavirus expression vector further comprises an alphavirus subgenomic promoter operatively linked to said nucleic acid sequence.

14. The vaccine of claim 1, wherein said packaging-defective recombinant molecule is produced by a method comprising operatively linking a nucleic acid sequence encoding a protective compound to a packaging-defective alphavirus expression vector capable of directing expression of said packaging-defective recombinant molecule to obtain a packaging-defective recombinant molecule.

15. The vaccine of claim 1, wherein said packaging-defective recombinant molecule is selected from the group consisting of SV1:nP30.1008, SV1:nP30.924, SV1:nP30.873, SV1:nP30.789, SV1:nP30.771, SV1:nP30.924SS, SV1:nP30.867SS, SV1:nP30.783SS, SV1:nP30.771SS, SV2:nP30.1008, SV2:nP30.924, SV2:nP30.873, SV2:nP30.789, SV2:nP30.771, SV2:nP30.924SS, SV2:nP30.867SS, SV2:nP30.783SS, and SV2:nP30.771SS.

16. The vaccine of claim 1, wherein said vaccine is produced by a method comprising:
(a) co-transfecting a host cell with said packaging-defective recombinant molecule and an alphavirus packaging vector, said packaging vector being able to effect packaging of said packaging-defective recombinant molecule into a recombinant virus particle comprising said packaging-defective recombinant molecule packaged into an alphavirus coat, said packaging vector being essentially incapable of self-packaging;
(b) culturing said transfected cell in an effective medium to produce said recombinant virus particle;
(c) recovering said recombinant virus particle; and
(d) formulating a vaccine therefrom.

17. The vaccine of claim 16, wherein said packaging vector comprises PV1.

18. The vaccine of claim 1, wherein said vaccine is produced by a method comprising:
(a) transfecting a host cell with said packaging-defective recombinant molecule, said host cell being capable of packaging said packaging-defective recombinant molecule into a recombinant virus particle comprising said packaging-defective recombinant molecule packaged into an alphavirus coat;
(b) culturing said transfected cell in an effective medium to produce said recombinant virus particle;
(c) recovering said recombinant virus particle; and
(d) formulating a vaccine therefrom.

19. The vaccine of claim 1, wherein said nucleic acid sequence is selected from the group consisting of nP30.1008, nP30.924, nP30.873, nP30.789, nP30.771, nP30.924SS, nP30.867SS, nP30.783SS and nP30.771SS.

20. The vaccine of claim 1, wherein said vaccine is selected from the group consisting of VPV SV1:nP30.1008, VPV SV1:nP30.924, VPV SV1:nP30.873, VPV SV1:nP30.789, VPV SV1:nP30.771, VPV SV1:nP30.924SS, VPV SV1:nP30.867SS, VPV SV1:nP30.783SS, VPV SV1:nP30.771SS, VPV SV2:nP30.1008, VPV SV2:nP30.924, VPV SV2:nP30.873, VPV SV2:nP30.789, VPV SV2:nP30.771, VPV SV2:nP30.924SS, VPV SV2:nP30.867SS, VPV SV2:nP30.783SS, and VPV SV2:nP30.771SS.

21. The vaccine of claim 1, further comprising a therapeutic composition comprising said protective compound.

22. An isolated nucleic acid sequence encoding a modified Toxoplasma gondii P30 antigen selected from the group consisting of a P30 antigen lacking amino terminal hydrophobic residues, a P30 antigen lacking carboxyl terminal hydrophobic residues, and a P30 antigen lacking both amino terminal and carboxyl terminal hydrophobic residues.

23. The nucleic acid sequence of claim 22, wherein said nucleic acid encodes a modified antigen further comprising a signal segment, a fusion segment, or a combination thereof.

24. The nucleic acid sequence of claim 22, wherein said nucleic acid sequence is selected from the group consisting of nP30.924, nP30.873, nP30.789, nP30.771, nP30.924SS, nP30.867SS, nP30.783SS, nP30.771SS.

25. A modified T. gondii P30 antigen selected from the group consisting of a P30 antigen lacking amino terminal hydrophobic residues, a P30 antigen lacking carboxyl terminal hydrophobic residues, and a P30 antigen lacking both amino terminal and carboxyl terminal hydrophobic residues.

26. The modified antigen of claim 25, wherein said modified antigen is capable of protecting an animal from toxoplasmosis when said antigen is administered to said animal in an effective amount.

27. The modified antigen of claim 25, wherein said modified antigen is capable of diagnosing infection by T. gondii.

28. The modified antigen of claim 25, wherein said modified antigen comprises a signal segment, a fusion segment, or a combination thereof.

29. The modified antigen of claim 25, wherein said modified antigen is selected from the group consisting of P30.336, P30.308, P30.291, P30.289, P30.263, P30.261, P30.257, GST-P30.336, GST-P30.308, GST-P30.291, GST-P 30.289, GST-P30.263, GST-P30.261 and GST-P30.257.

30. A packaging vector comprising PV1.

31. A method to protect an animal from disease, comprising administering to said animal an effective amount of a recombinant virus particle vaccine having a packaging-defective recombinant molecule packaged in an alphavirus coat, said packaging-defective recombinant molecule comprising a nucleic acid sequence that encodes at least one protective compound, said nucleic acid sequence being operatively linked to a packaging-defective alphavirus expression vector capable of directing expression of said packaging-defective recombinant molecule, said animal being capable of expressing said packaging-defective recombinant molecule.

32. The method of claim 31, wherein said disease is caused by an infectious agent selected from the group consisting of protozoan parasites, helminth parasites, ectoparasites, fungi, bacteria, and viruses.

33. The method of claim 31, wherein said animal is selected from the group consisting of mammals, insects, and birds.

34. The method of claim 31, wherein said sequence encodes a protective compound comprising a Toxoplasma gondii P30 antigen.

35. The method of claim 31, further comprising administering to said animal a substantially pure protective compound prior to, following, or both prior to and following administering said recombinant virus particle vaccine, said protective compound being encoded by said nucleic acid sequence.

36. A method to produce a recombinant virus particle vaccine, comprising:
(a) co-transfecting a host cell with a packaging-defective recombinant molecule and an alphavirus packaging vector, wherein said packaging-defective recombinant molecule comprises a nucleic acid sequence that encodes at least one protective compound, said nucleic acid sequence being operatively linked to a packaging-defective alphavirus expression vector, wherein said packaging vector is able to effect packaging of said packaging-defective recombinant molecule into a recombinant virus particle comprising said packaging-defective recombinant molecule packaged into an alphavirus coat, and wherein packaging vector is essentially incapable of self-packaging;
(b) culturing said transfected cell in an effective medium to produce said recombinant virus particle;
(c) recovering said recombinant virus particle; and
(d) formulating a vaccine therefrom.

37. The method of claim 36, wherein said alphavirus packaging vector comprises PV1.

38. A method to produce a recombinant virus particle vaccine, comprising:
(a) transfecting a host cell with a packaging-defective recombinant molecule, wherein said packaging-defective recombinant molecule comprises a nucleic acid sequence that encodes at least one protective compound, said nucleic acid sequence being operatively linked to a packaging-defective alphavirus expression vector, and wherein said host cell is capable of packaging said packaging-defective recombinant molecule into a recombinant virus particle comprising said packaging-defective recombinant molecule packaged into an alphavirus coat;
(b) culturing said transfected cell in an effective medium to produce said recombinant virus particle;
(c) recovering said recombinant virus particle; and
(d) formulating a vaccine therefrom.

39. A method to protect an animal from toxoplasmosis comprising administering to said animal an effective amount of a recombinant virus particle vaccine comprising a packaging-defective recombinant molecule packaged in an alphavirus coat, said packaging-defective recombinant molecule comprising a nucleic acid sequence encoding a Toxoplasma gondii P30 antigen operatively linked to a packaging-defective Sindbis virus expression vector capable of directing replication and expression of said packaging-defective recombinant molecule.

40. A recombinant molecule capable of directing expression of at least one protective compound when said recombinant molecule is transfected into an animal host cell, said recombinant molecule comprising a nucleic acid sequence encoding at least one protective compound selected from the group consisting of a protective protein and a protective RNA species, said nucleic acid sequence being operatively linked to an alphavirus expression vector, said compound being capable of protecting an animal from disease caused by a parasite.

41. The recombinant molecule of claim 40, wherein said parasite is selected from the group consisting of a protozoan, a helminth, an ectoparasite, a fungus, a bacterium, a virus and a combination thereof.

42. The recombinant molecule of claim 40, wherein said parasite is selected from the group consisting of Toxoplasma, Dirofilaria, Acanthocheilonema, Babesia, Brugia, Candida, Cryptococcus, Cryptosporidium, Dipetalonema, Eimeria, Encephalitozoon, Hepatozoon, Histoplasma, Isospora, Loa, Microsporidia, Neospora, Nosema, Onchocerca, Parafilaria, Plasmodium, Pneumocystis, Rochalimaea, Setaria, Stephanofilaria, Theileria and Wuchereria.

43. The recombinant molecule of claim 40, wherein said parasite is selected from the group consisting of Toxoplasma, Dirofilaria and a combination thereof.

44. The recombinant molecule of claim 40, wherein said protective compound comprises a T. gondii P30 antigen.

45. The recombinant molecule of claim 44, wherein said T. gondii P30 antigen comprises a modified T. gondii P30 antigen selected from the group consisting of a P30 antigen lacking amino terminal hydrophobic residues, a P30 antigen lacking carboxyl terminal hydrophobic residues and a P30 antigen lacking both amino terminal and carboxyl terminal hydrophobic residues.

46. The recombinant molecule of claim 44, wherein said T. gondii P30 antigen is selected from the group consisting of P30.336, P30.308, P30.291, P30.289, P30.263, P30.261, P30.257, GST-P30.336, GST-P30.308, GST-P30.291, GST-P30.289, GST-P30.263, GST-P30.261, GST-P30.257, and a combination thereof.

47. The recombinant molecule of claim 40, wherein said protective protein further comprises a fusion segment.

48. The recombinant molecule of claim 47, wherein said fusion segment is selected from the group consisting of a glutathione binding domain, a metal binding domain, an immunoglobulin binding domain and a sugar binding domain.

49. The recombinant molecule of claim 40, wherein said protective protein further comprises a signal segment capable of promoting secretion of said protective protein.

50. The recombinant molecule of claim 49, wherein said signal segment is selected from the group consisting of tissue plasminogen activator, interferon, interleukin, growth hormone, histocompatibility and viral signal segments.

51. The recombinant molecule of claim 40, wherein said alphavirus expression vector is selected from the group consisting of a Sindbis virus expression vector, a Semliki Forest virus expression vector, a Ross River virus expression vector, a Venezuela equine encephalitis virus expression vector or wherein said alphavirus expression vector comprises a hybrid of any one or more of said alphavirus expression vectors.

52. The recombinant molecule of claim 40, wherein said alphavirus expression vector is selected from the group consisting of SV1, SV2, SV3, SV4, SV5 and SV6.

53. The recombinant molecule of claim 40, wherein said alphavirus expression vector further comprises an alphavirus subgenomic promoter operatively linked to said nucleic acid sequence.

54. The recombinant molecule of claim 40, wherein said alphavirus expression vector comprises a Sindbis virus expression vector having a Sindbis virus subgenomic promoter and wherein said nucleic acid sequence encodes a T. gondii P30 antigen.

55. The recombinant molecule of claim 54, wherein said antigen further comprises a signal segment capable of promoting secretion of said protective protein.

56. The recombinant molecule of claim 55, wherein said signal segment comprises a tissue plasminogen activator signal segment.

57. The recombinant molecule of claim 54, wherein said antigen further comprises a fusion segment.

58. The recombinant molecule of claim 57, wherein said fusion segment is selected from a glutathione binding domain and a poly-histidine segment.

59. The recombinant molecule of claim 40, wherein said nucleic acid sequence is selected from the group consisting of nP30.1008, nP30.924, nP30.873, nP30.789, nP30.771, nP30.924SS, nP30.867SS, nP30.783SS, nP30.771SS, nGST-nP30.1008, nGST-nP30.924, nGST-nP30.873, nGST-nP 30.789, nGST-nP30.771, nGST-nP30.924SS, nGST-nP30.867SS, nGST-nP30.783SS, nGST-nP30.771SS and combinations thereof.

60. The recombinant molecule of claim 40, wherein said recombinant molecule is selected from the group consisting of an alphavirus expression vector selected from the group consisting of SV1, SV2, SV3, SV4, SV5 and SV6 operatively linked to at least one nucleic acid sequence selected from the group consisting of nP30.1008, nP30.924, nP30.873, nP30.789, nP30.771, nP30.924SS, nP30.867SS, nP30.783SS, nP30.771SS, nGST-nP30.1008, nGST-nP30.924, nGST-nP30.873, nGST-nP30.789, nGST-nP30.771, nGST-nP 30.924SS, nGST-nP30.867SS, nGST-nP30.783SS and nGST-nP 30.771SS.

61. The recombinant molecule of claim 40, wherein said animal host cell is selected from the group consisting of a mammalian cell, an insect cell, and an avian cell.

62. A recombinant molecule comprising an alphavirus expression vector operatively linked to a nucleic acid sequence encoding a fusion protein, said fusion protein comprising a fusion segment joined to a protein heterologous to said alphavirus.

63. A recombinant cell capable of producing at least one compound capable of protecting an animal from disease caused by a parasite, said cell comprising an animal cell transfected with a recombinant molecule capable of directing expression of said protective compound, said recombinant molecule comprising a nucleic acid sequence capable of encoding at least one protective compound operatively linked to an alphavirus expression vector.

64. The recombinant cell of claim 63, wherein said animal cell is selected from the group consisting of mammalian, insect and avian cells.

65. The recombinant cell of claim 63, wherein said recombinant cell is transfected with at least one recombinant molecule comprising an alphavirus expression vector selected from the group consisting of SV1, SV2, SV3, SV4, SV5 and SV6 operatively linked to at least one nucleic acid sequence selected from the group consisting of nP30.1008, nP30.924, nP30.873, nP30.789, nP30.771, nP30.924SS, nP30.867SS, nP30.783SS, nP30.771SS, nGST-nP 30.1008, nGST-nP30.924, nGST-nP30.873, nGST-nP30.789, nGST-nP30.771, nGST-nP30.924SS, nGST-nP30.867SS, nGST-nP 30.783SS and nGST-nP30.771SS.

66. A therapeutic composition capable of protecting an animal from disease caused by a parasite when said composition is administered to said animal in an effective amount, said composition being produced by a method comprising:
(a) culturing an animal cell transfected with a recombinant molecule to produce at least one compound capable of protecting said animal from said disease, said recombinant molecule comprising a nucleic acid sequence capable of encoding at least one protective compound operatively linked to an alphavirus expression vector, said compound being selected from the group consisting of a protective protein and a protective RNA species;
(b) recovering said protective compound; and
(c) formulating a therapeutic composition therefrom.

67. The composition of claim 66, wherein said protective protein further comprises a fusion segment.

68. The composition of claim 66, wherein said protective protein further comprises a signal segment capable of promoting secretion of said antigen from said transfected cell.

69. The composition of claim 66, wherein said composition further comprises an immunopotentiator.

70. The composition of claim 66, wherein said protective compound is selected from the group consisting of P30.336, P30.308, P30.291, P30.289, P30.263, P30.261, P30.257, GST-P30.336, GST-P30.308, GST-P30.291, GST-P 30.289, GST-P30.263, GST-P30.261, GST-P30.257, and combinations thereof.

71. A method to produce a therapeutic composition capable of protecting an animal from disease caused by a parasite, said method comprising:
(a) culturing an animal cell transfected with a recombinant molecule to produce at least one compound capable of protecting said animal from said disease, said recombinant molecule comprising a nucleic acid sequence capable of encoding at least one protective compound operatively linked to an alphavirus expression vector;
(b) recovering said protective compound; and
(c) formulating a therapeutic composition therefrom.

72. A method to protect an animal from a disease caused by a parasite comprising administering to said animal an effective amount of a therapeutic composition produced by a method comprising:
(a) culturing an animal cell transfected with a recombinant molecule to produce a compound capable of protecting said animal from said disease, said recombinant molecule comprising a nucleic acid sequence capable of encoding at least one protective compound operatively linked to an alphavirus expression vector;
(b) recovering said protective compound; and
(c) formulating a therapeutic composition therefrom.

73. The method of claim 72, wherein said animal is selected from the group consisting of mammals, insects, and birds.

74. The method of claim 72, wherein said composition is capable of protecting said animal from toxoplasmosis or heartworm.

75. A method to protect an animal from toxoplasmosis comprising administering to said animal an effective amount of a therapeutic composition produced by a method comprising:
(a) culturing an animal cell transfected with a recombinant molecule to produce a T. gondii P30 antigen, said recombinant molecule comprising at least one nucleic acid sequence encoding said antigen operatively linked to an alphavirus expression vector;
(b) recovering said antigen; and
(c) formulating a therapeutic composition therefrom.

76. The method of claim 63, wherein said antigen comprises a modified T. gondii P30 antigen selected from the group consisting of a P30 antigen lacking amino terminal hydrophobic residues, a P30 antigen lacking carboxyl terminal hydrophobic residues, and a P30 antigen lacking both amino terminal and carboxyl terminal hydrophobic residues.

77. A method to produce a recombinant molecule comprising operatively linking a nucleic acid sequence capable of encoding at least one protective compound to an alphavirus expression vector capable of directing expression of said recombinant molecule to obtain a recombinant molecule.


SEQUENCE LISTING (13 sequences)

SEQ ID NO: 1 - DNA, 1011 bp, Toxoplasma gondii
  CDS (1)..(1011)
atgtcggtttcgctgcaccacttcattatttcttctggttttttgacgagtatgtttccgaaggcagtgagacgcgccgtcacggcaggggtgtttgccgcgcccacactgatgtcgttcttgcgatgtggcgttatggcatcggatccccctcttgttgccaatcaagttgtcacctgcccagataaaaaatcgacagccgcggtcattctcacaccgacggagaaccacttcactctcaagtgccctaaaacagcgctcacagagcctcccactcttgcgtactcacccaacaggcaaatctgcccagcgggtactacaagtagctgtacatcaaaggctgtaacattgagctccttgattcctgaagcagaagatagctggtggacgggggattctgctagtctcgacacggcaggcatcaaactcacagttccaatcgagaagttccccgtgacaacgcagacgtttgtggtcggttgcatcaagggagacgacgcacagagttgtatggtcacggtgacagtacaagccagagcctcatcggtcgtcaataatgtcgcaaggtgctcctacggtgcagacagcactcttggtcctgtcaatttgtctgcggaaggacccactacaatgaccctcgtgtgcgggaaagatggagtcaaagttcctcaagacaacaatcagtactgttccgggacgacgctgactggttgcaacgagaaatcgttcaaagatattttgccaaaattaactgagaacccgtggcagggtaacgcttcgagtgataagggtgccacgctaacgatcaagaaggaagcatttccagccgagtcaaaaagcgtcattattggatgcacagggggatcgcctgagaagcatcactgtaccgtgaaactggagtttgccggggctgcagggtcagcaaaatcggctgcgggaacagccagtcacgtttccatttttgccatggtgatcggacttattggctctatcgcagcttgtgtcgcgtga

SEQ ID NO: 2 - protein, 336 aa, Toxoplasma gondii
MSVSLHHFIISSGFLTSMFPKAVRRAVTAGVFAAPTLMSFLRCGVMASDPPLVANQVVTCPDKKSTAAVILTPTENHFTLKCPKTALTEPPTLAYSPNRQICPAGTTSSCTSKAVTLSSLIPEAEDSWWTGDSASLDTAGIKLTVPIEKFPVTTQTFVVGCIKGDDAQSCMVTVTVQARASSVVNNVARCSYGADSTLGPVNLSAEGPTTMTLVCGKDGVKVPQDNNQYCSGTTLTGCNEKSFKDILPKLTENPWQGNASSDKGATLTIKKEAFPAESKSVIIGCTGGSPEKHHCTVKLEFAGAAGSAKSAAGTASHVSIFAMVIGLIGSIAACVA

SEQ ID NO: 3 - DNA, 33 bp, Artificial sequence
  Synthetic Primer
cccgggtctagaatgtcggtttcgctgcaccac

SEQ ID NO: 4 - DNA, 33 bp, Artificial sequence
  Synthetic Primer
acgcgtggtacctcgagtcacgcgacacaagct

SEQ ID NO: 5 - DNA, 37 bp, Artificial sequence
  Synthetic Primer
gtcgaccccgggtctagaccatggcatcggatccccc

SEQ ID NO: 6 - DNA, 41 bp, Artificial sequence
  Synthetic Primer
acgcgtggtacctcgagttatgctgaccctgcagccccggc

SEQ ID NO: 7 - DNA, 35 bp, Artificial sequence
  Synthetic Primer
gtcgactctagacccgggatggcatcggatccccc

SEQ ID NO: 8 - DNA, 34 bp, Artificial sequence
  Synthetic Primer
acgcgtggtaccgaattctcacgcgacacaagct

SEQ ID NO: 9 - DNA, 110 bp, Artificial sequence
  Synthetic Primer
gtcgaccccgggtctagaccatggatgcaatgaagagagggctctgctgtgtgctgctactgtgtggagcagtcttcgtttcgcccagctcggatccccctcttgttgcc

SEQ ID NO: 10 - DNA, 37 bp, Artificial sequence
  Synthetic Primer
gtcgaccccgggtctagaccatgtcccctatactagg

SEQ ID NO: 11 - DNA, 33 bp, Artificial sequence
  Synthetic Primer
acgcgtggtacctcgagtcagtcagtcacgatg

SEQ ID NO: 12 - DNA, 40 bp, Artificial sequence
  Synthetic Primer
gtcgaccccgggtctagaaccatggctctcagtgaaatca

SEQ ID NO: 13 - DNA, 37 bp, Artificial sequence
  Synthetic Primer
acgcgtggtacctcgagtcatctgcagccttcttgaa